(19) United States
(12) Patent Application Publication
(10) Pub. No.: US 2026/0069694 A1
(43) Pub. Date: Mar. 12, 2026

(54) IDENTIFICATION OF HIGHLY IMMUNOSUPRESIVE TREG POPULATION IN TUMOR MICROENVIRONMENTS

(71) Applicant: THE TRUSTEES OF COLUMBIA UNIVERSITY IN THE CITY OF NEW YORK, New York, NY (US)

(72) Inventors: Kazuki SUGAHARA, Fort Lee, NJ (US); Yuki KUNISADA, Okayama (JP); Kodai SUZUKI, Gifu (JP); Norio MIYAMURA, Fort Lee, NJ (US)

(21) Appl. No.: 19/387,807

(22) Filed: Nov. 13, 2025

Related U.S. Application Data
(63) Continuation of application No. PCT/US24/30209, filed on May 20, 2024.
(60) Provisional application No. 63/503,392, filed on May 19, 2023.

(51) Int. Cl. A61K 40/11; A61K 38/08; A61K 38/17; A61K 39/00; A61K 39/395; A61K 40/22; A61K 40/50; A61P 35/00; A61P 37/06; C12N 5/00; C12N 5/0783

(52) U.S. Cl. CPC A61K 40/11; A61K 38/08; A61K 38/1777; A61K 39/39541; A61K 40/22; A61K 40/50; A61P 35/00; A61P 37/06; C12N 5/0081; C12N 5/0637; A61K 2039/505; C12N 2501/15; C12N 2501/2302; C12N 2501/515; C12N 2501/599; C12N 2502/30

(57) ABSTRACT

Pancreatic ductal adenocarcinoma (PDAC) has abundant immunosuppressive regulatory T cells (Tregs) which contribute to a tumor microenvironment that is resistant to immunotherapy. Tregs in the PDAC tissue, but not those in the spleen, express the αvβ5 integrin in addition to neuropilin-1 (NRP-1), which makes them susceptible to the iRGD tumor-penetrating peptide that targets αv integrin- and NRP1-positive cells. As a result, long-term treatment of PDAC mice with iRGD leads to a tumor-specific decrease of Tregs and improved efficacy of an immune checkpoint blockade. αvβ5 integrin+ Tregs are induced from both naïve CD4+ T cells and natural Tregs upon T cell receptor stimulation, and represent a highly immunosuppressive subpopulation of CCR8+ Tregs. This study identifies αvβ5 integrin as a marker for activated tumor-resident Tregs that can be expanded to achieve tumor-specific Treg depletion to improve anti-tumor immunity for PDAC management.

### CROSS-REFERENCE TO RELATED APPLICATIONS

This application is an international application which claims the benefit of U.S. provisional application Ser. No. 63/503,392, filed 19 May 2023. The entire contents of this application is hereby incorporated by reference as if fully set forth herein.

### GOVERNMENT FUNDING SUPPORT

This invention was made with government support under W81XWH-20-1-0887, awarded by the Medical Research and Development Command and under grant no. CA167174, awarded by the National Institutes of Health. The government has certain rights in the invention.

### BACKGROUND OF THE INVENTION

### 1. Field of the Invention

The invention relates to the general field of biomedicine. Specifically, the invention relates to tumor-resident regulatory T cells in pancreatic cancers that express αvβ5 integrin which can be used as a targetable activation marker.

### 2. Background of the Invention

Pancreatic ductal adenocarcinoma (PDAC) is resistant to immunotherapy, at least in part, due to the prevalence of immunosuppressive cells and paucity of cytotoxic effector cells. Regulatory T cells (Tregs) are believed to be a significant driver of the immunosuppressive microenvironment in the PDAC tissue that protects the cancer cells from T cell mediated anti-tumor immunity. In fact, a decreased ratio of tumor-infiltrating CD8⁺ T cells to Tregs (CD8/Treg ratio) has been shown to correlate with poor prognosis in PDAC patients and diminished response to immune checkpoint blockades (ICBs).

Various approaches have been attempted to deplete tumor-resident Tregs to improve the CD8/Treg ratio and sensitize PDAC to ICB. Examples include inhibitors of cytokines and chemokines that recruit Tregs to PDAC tissue, antibodies (Abs) that target cell surface molecules expressed on Tregs such as CD25, cytotoxic T-lymphocyte-associated protein 4 (CTLA-4), programmed cell death-1 (PD-1), and the glucocorticoid induced TNF receptor. Small molecules such as cyclophosphamide that kill highly proliferating cells have also been used. However, most of the approaches lead to systemic Treg depletion, which can potentially result in serious autoimmune diseases. A study using transgenic Kras^{+/LSL-G12D}; Ptf1a^{+/Cre}; Foxp3^{tm3(DTR/GFP)Ayr} (KC; Foxp3^{DTR}) mice showed that systemic Treg depletion led to an unexpected acceleration of tumor progression, which was caused by the induction of inflammatory fibroblasts and chemokines that drove tumor infiltration of immunosuppressive myeloid cells. These findings suggest that Treg depletion must be achieved in a controlled manner, ideally in a tumor-specific fashion, to avoid autoimmune complications and unwanted alterations of the tumor immune microenvironment (TIME).

### SUMMARY OF THE INVENTION

Thus, there exists a need in the art for controlled and tumor-specific Treg depletion.

In particular embodiments, the present invention relates to a method of obtaining a highly immunosuppressive Treg population comprising: (a) obtaining a tumor sample from a subject; and (b) isolating from the tumor sample CCR8+ Treg cells that express αvβ5 integrin. In a preferred embodiment, the tumor sample is a pancreatic tumor sample.

In other particular embodiments, the present invention relates to a method of obtaining a highly immunosuppressive Treg population comprising: (a) obtaining a sample of CD4+ cells from a subject; (b) contacting the CD4+ cells to a reagent selected from the group consisting of one or more of anti-CD3 antibody, anti-CD28 antibody, anti-CD3/CD28 bispecific antibody and TGFβ1 under conditions to bind a population of CD4+Foxp3+ Tregs; and (c) isolating the CD4+Foxp3+ Tregs from the population of cells, wherein the CD4+Foxp3+ Tregs express αvβ5 integrin. Preferably, the isolated CD4+Foxp3+ Tregs are CCR8+ cells that express αvβ5 integrin.

In another embodiment, the invention related to an isolated population of αvβ5 integrin expressing CCR8+ Treg cells, to an isolated population of αvβ5 integrin expressing CCR8+ Treg cells obtained by a method above, and to an isolated population of αvβ5 integrin expressing CD4+Foxp3+ Tregs obtained by a method above.

In a further embodiment, the invention relates to a method for reducing immune responses in a subject in need, the method comprising administering to the subject an effective composition comprising αvβ5 integrin expressing CCR8+ Treg cells as described above and herein or comprising αvβ5 integrin expressing CCR8+ Treg cells as described above and herein. Preferable, for these embodiments of the invention, the subject in need is a subject who has symptoms of an autoimmune disorder or has received a tissue transplant or who is experiencing graft-versus-host disease.

Additional embodiments involve also exposing a graft material to αvβ5 integrin expressing CCR8+ Treg cells prior to transplantation of the graft material.

Further embodiments of the invention relate to a method of treating cancer in a subject in need, comprising: administering to the subject in need an αvβ5 integrin inhibitor in an amount effective to reduce αvβ5 integrin expression in the subject in need. Preferably, the αvβ5 integrin inhibitor comprises an αvβ5 integrin specific antibody, an αvβ5 affinity probe, an αvβ5 integrin specific aptamer, a small molecule αvβ5 integrin inhibitor, or an αvβ5 integrin inhibitory oligonucleotide. Such method optionally also can include co-administering one or more immune checkpoint inhibitors to the subject in need. The one or more immune checkpoint inhibitors can be selected from the group consisting of a PD-1 inhibitor, a PD-L1 inhibitor, a PD-L2 inhibitor, or any combination thereof.

### BRIEF SUMMARY OF THE DRAWINGS

Certain embodiments are illustrated by way of example, and not by way of limitation, in the figures of the accompanying drawings.

FIG. 1A is a set of representative images of CD8+ T cells in the PDAC tissue (brown arrowheads show examples).

FIG. 1B is a bar diagram showing the number of CD8+ T cells per high-power field (n=3) per arm.

FIG. 2A shows representative images of Foxp3+ Tregs (red) in PDAC tissue.

FIG. 2B is a bar diagram showing the number of Foxp3+ Tregs per high-power field in the primary PDAC tumors.

FIG. 3 shows representative images showing longitudinal growth of an orthotopic luciferase+ KPC-derived PDAC tumor in a mouse with a matching genetic background analyzed by bioluminescence imaging.

FIG. 4A and FIG. 4B present data for C57B6129SF1/J hybrid mice bearing orthotopic KPC-derived PDAC tumors that were treated with PBS 3 times a week for 2 weeks.

FIG. 4C shows data on the CD8/Treg ratio, calculated based on the values from FIG. 4A and FIG. 4B

FIG. 5A is a bar diagram showing results on tumor growth.

FIG. 5B shows representative images of the tumors.

FIG. 6A and FIG. 6B present flow cytometry results in tumor and spleen, respectively.

FIG. 7A and FIG. 7B are bar diagrams presenting data on the % Tregs in CD4+ T cells in PDAC and the CD8/Tregs ratio in PDAC, respectively.

FIG. 8A and FIG. 8B show representative images of IF performed on PDAC tissue.

FIG. 8C shows the CD8/Treg ratio.

FIG. 9A and FIG. 9B are bar diagrams presenting data on the % Tregs in CD4+ T cells in spleen, and the CD8/Tregs ratio in spleen, respectively.

FIG. 10A shows flow cytometry analysis results.

FIG. 10B shows representative immunofluorescence images showing the expression of PD-L1 (green) in orthotopic KPC-derived PDAC tissue.

FIG. 11A, FIG. 11B and FIG. 11C show results for tumor weight.

FIG. 12A and FIG. 12B are representative confocal images of αvβ5 integrin+ CD4+ Foxp3⁺ T cells and NRP-1+CD4+Foxp3+ T cells, respectively, in the PDAC tissue of KPC-derived orthotopic PDAC mice.

FIG. 13A, FIG. 13B, and FIG. 13C present results from flow cytometry analysis showed the proportion of CD4+CD25+ Tregs positive for αvβ5 integrin, NRP-1, or both in the spleen and PDAC in KPC derived orthotopic PDAC mice.

FIG. 14A, FIG. 14B, and FIG. 14C present results from flow cytometry analysis showing the proportion of CD8+ T cells, CD4+CD25neg T cells (non-Tregs), and CD4+CD25+ Tregs that are positive for αvβ5 integrin, NRP-1, or both in the PDAC of KPC-derived orthotopic PDAC mice.

FIG. 15A presents flow cytometry results for the indicated cells.

FIG. 15B shows representative confocal images of αvβ5 integrin+Foxp3+CD4+ Tregs and NRP-1+CD4+ Tregs in human PDAC and spleen.

FIG. 15C presents flow cytometry results for the indicated cells.

FIG. 15D presents flow cytometry results for the indicated cells as a percentage of MAX (naïve CD4⁺ T cells, and human Tregs).

FIG. 16A shows the expression of αvβ5 integrin and NRP-1 on CD4+CD25+ Tregs expanded with or without the PDAC cells (n=4 per group.

FIG. 16B shows the expression of αvβ5 integrin and NRP-1 on CD4+CD25+ Tregs and non-Treg CD4+CD25neg T cells expanded with the PDAC cells.

FIG. 17A and FIG. 17B show FAM-iRGD binding to the non-Tregs (blue line) and Tregs (red line) shown in FIG. 16B.

FIG. 18 shows the dose-dependent inhibition of FAMiRGD binding by an anti-αvβ5 integrin blocking Ab to Tregs that were expanded in the presence of PDAC cells.

FIG. 19 is a bar diagram showing apoptosis in Tregs and non-Tregs expanded in the presence of PDAC cells with or without iRGD.

FIG. 20A and FIG. 20B present data on in vitro binding of FAM-iRGD to non-Treg CD4+CD25neg T cells (blue line) and CD4+CD25+ Tregs (red line) isolated from the PDAC tissue of KPC-derived orthotopic PDAC mice.

FIG. 21A and FIG. 21B present data on in vitro binding of FAM-iRGD to non-Treg CD4+CD25neg T cells (blue line) and CD4+CD25+ Tregs (red line) isolated from the spleen.

FIG. 22 shows representative confocal images of Foxp3+ T cells (red) in the PDAC tissue of KPC-derived orthotopic PDAC mice that received an intravenous injection of FAM-iRGD.

FIG. 23A. FIG. 23B, FIG. 23C, and FIG. 23D show the analysis for αvβ5 integrin and NRP-1 expression by flow cytometry.

FIG. 24A, FIG. 24B, and FIG. 24C are bar graphs showing data on NRP1 expression in the indicated cells.

FIG. 25A shows a flow cytometric analysis showing the proportion of CD4+Foxp3+ T cells (iTregs) after the expansion.

FIG. 25B is a bar diagram that summarizes the results from 3 independent experiments from FIG. 25A.

FIG. 26A shows the expression of αvβ5 integrin (red boxes) and NRP-1 (blue boxes) on the iTregs gated in 25A.

FIG. 26B is a bar diagram that summarizes the results from 3 independent experiments from FIG. 26A.

FIG. 27A provides cytometry data for populations of cells gated based on Foxp3 and CD25 expression.

FIG. 27B and FIG. 27C are bar diagrams summarize the proportion of αvβ5 integrin-positive cells in the indicated T cell population.

FIG. 28A presents flow cytometry data for cell populations.

FIG. 28B is a bar diagram that summarizes the proportion of αvβ5 integrin-positive cells in the indicated T cell population.

FIG. 29A, FIG. 29B, and FIG. 29C are bar graphs that show the proportion of NRP-1+ cells among CD25-positive and negative populations in CD4+Foxp3+ T cells (FIG. 29A) and CD4+Foxp3neg T cells (FIG. 29B and FIG. 29C) after treatment.

FIG. 30 shows flow cytometric analysis showing the proportion of αvβ5 integrin+ cells among CD4+CD25+Foxp3+ iTregs induced by increasing concentrations of anti-CD3 Ab.

FIG. 31 shows results from flow cytometry performed to quantify the proportion of CD4+CD25+Foxp3+ iTregs.

FIG. 32A shows results from flow cytometry performed to quantify the proportion of αvβ5 integrin+ cells among iTregs.

FIG. 32B shows quantitation of apoptosis of αvβ5 integrin+ iTregs quantitated by measuring cleaved caspase 3 using flow cytometry.

FIG. 33A shows results from flow cytometry analysis of the expression of CD25 and Foxp3 in the CD4+ T cells.

FIG. 33B shows results from flow cytometry analysis of the expression of the αvβ5 integrin and NRP-1 on CD4+CD25+Foxp3+ iTregs.

FIG. 33C is a bar diagram that summarizes the proportion of NRP-1+ cells among the iTregs from FIG. 33A and FIG. 33B.

FIG. 34 shows flow cytometry results for the proportion of apoptotic αvβ5 integrin+ iTregs.

FIG. 35 shows flow cytometry results for the proportion of CD4+Foxp3neg T cells.

FIG. 36A shows the expression of αvβ5 integrin and NRP-1 on the nTregs (red box) and naïve CD4+CD25neg Foxp3neg T cells (blue box) analyzed by flow cytometry.

FIG. 36B is a bar diagram that summarizes the proportion of αvβ5 integrin+ cells among the two populations from FIG. 36A.

FIG. 37A shows flow cytometry results from the pool in (FIG. 36A) treated with anti-CD3/CD28 Abs alone for 3 days (see FIG. 37A, left panel).

FIG. 37B and FIG. 37C are bar graphs that summarize the data in FIG. 37A.

FIG. 38A presents flow cytometry results showing that naïve CD4⁺ CD25^{neg} Foxp3neg T cells did not express CCR8 (or αvβ5 integrin).

FIG. 38B shows representative dot plots from 3 or 4 separate studies of the expression of CCR8 and αvβ5 integrin on T cells.

FIG. 38C is a bar graph summarizing the proportion of CCR8+ cells and αvβ5 integrin+ cells among each T cell population in FIG. 38A and FIG. 38B.

FIG. 39 is a Venn diagram summarizing the proportion of Tregs that express CCR8 and/or αvβ5 integrin based on the results from FIG. 38.

FIG. 40A and FIG. 40B show dot plot data for the induced iTregs from mouse naïve CD4⁺ T cells, and magnetically enriched for CCR8+ iTregs.

FIG. 40C and FIG. 40D show dot plot data for CCR8+ iTregs that were either depleted or enriched for αvβ5 integrin+ cells as indicated.

FIG. 40E is a set of bar diagrams that summarize the values from FIG. 40A through FIG. 40D, normalized to stimulated Tconv 967 alone.

FIG. 41A, FIG. 41B, and FIG. 41C are bar graphs that show NRP-1 expression on nTregs and CD4⁺ CD25neg Foxp3^{neg} T cells prior to treatment.

### DETAILED DESCRIPTION OF EMBODIMENTS OF THE INVENTION

### 1. Overview

The work presented here revealed that the expression of αvβ5 integrin in the tumor (in addition to NRP-1) is critical for iRGD to effectively penetrate the extravascular tumor tissue.

Previous studies have shown that the αvβ5 integrin is expressed in about 80% of human PDAC tissues and is a predictor of poor prognosis in PDAC patients. In fact, epithelial cells and cancer-associated fibroblasts (CAFs) in the PDAC tissue often express high levels of αvβ5 integrin and NRP-1, and their crosstalk mediated by transforming growth factor-β (TGF-β) helps maintain the expression of αvβ5 integrin, creating a tumor microenvironment (TME) that is optimal for iRGD penetration. Therefore, iRGD co-injection therapy is highly effective against PDAC despite the desmoplastic nature of the tumor.

In a recent phase 1b clinical trial, iRGD (in the name of CEND-1) showed promising preliminary efficacy in combination with gemcitabine (Gem) and nab-paclitaxel (Nab-P) against metastatic PDAC. iRGD is now being evaluated in multiple phase 2 clinical trials for PDAC and other cancers (e.g., NCT05042128). Here, we report that the αvβ5 integrin is also expressed on Tregs that infiltrate the PDAC tissue, but not those in the spleen, allowing iRGD to selectively target PDAC-resident Tregs. Importantly, the αvβ5 integrin defines a highly immunosuppressive subpopulation of CCR8+ Tregs, providing a way to target activated Tregs in a tumor-specific manner.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As will be appreciated by those of ordinary skill in the art, the absolute amount of a particular agent that is effective may vary depending on such factors as the subject being treated, the biological endpoint, the particular active agent, the target tissue, etc. An effective amount of an agent or composition generally is an amount sufficient to achieve one or more of the following in a subject in need: a complete response (remission), a partial response, achievement of stable disease as determined by objective criteria, an improvement in symptoms, an increase in the length of progression-free survival, or an increase in overall survival. In the context of cancer, an effective amount can be an amount that results in killing of tumor cells, directly or indirectly or that stops growth of the tumor cells. In the context of an immune disorder, an effective amount is an amount that reduces immune responses within a subject whether the immune responses are innate to the subject or produced by graft material transplanted into a subject.

Those of ordinary skill in the art will further understand that an “effective amount” may be administered in a single dose, or may be achieved by administration of multiple doses over a period of time. An effective amount of a pharmaceutical composition that contains an effective amount of one or more agents is an amount of each agent such that the overall composition is effective.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0+0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the terms “subject,” “individual,” “host,” and “patient,” are used interchangeably to refer to humans or any non-human mammal, and can include mammalian laboratory animals, mammalian farm animals, mammalian sport animals, mammalian companion animals, simians, non-human primates, felines, canines, equines, rodents, lagomorphs, bovines, porcines, ovines, caprines, and the like. A suitable subject for the invention preferably is a human that is suspected of having, has been diagnosed as having, or is at risk of developing a hyperproliferative disease or an adverse immune response. Conditions amenable to treatment by the invention which define an appropriate subject or patient will be discerned easily by the person of skill in the art based on the disclosures herein. A “subject in need” is a subject that is at risk of developing cancer, or who manifests any characteristics or symptoms of cancer, or who has been diagnosed with cancer or is at risk of developing an immune disorder or who manifests any characteristics or symptoms of an immune disorder, or who has been diagnosed with immune disorder. Examples of immune disorders include autoimmune disorders and graft-versus-host disease.

As used herein, the term “antibody” refers to an immunoglobulin and encompasses full size antibodies and antibody fragments comprising an antigen binding site. Antibodies useful in certain embodiments of the invention may originate from or be derived from a mammal, e.g., a human, non-human primate, rodent (e.g., mouse, rat), rabbit, goat, bovine, equine, ovine, camelid, or from a bird (e.g., chicken), and may be of any of the various antibody isotypes, e.g., the mammalian isotypes: IgG (e.g., of the IgG1, IgG2, IgG3, or IgG4 subclass), IgM, IgA, IgD, and IgE or isotypes that are not found in mammals, e.g., IgY (found in birds) or IgW (found in sharks).

As used herein, the term “Treg” refers to T cells with regularory properties, for example immunosuppressive properties.

As used herein, the term “nTreg” refers to natural Treg cells that develop as a distinct lineage in the thymus.

As used herein, the term “iTreg” refers to induced Treg cells that arise from peripheral naïve conventional T cells.

### 3. Embodiments of the Invention

### A. Overview of Invention

The benefits of tumor-specific Treg depletion in cancer therapy have been demonstrated by targeting the chemokine receptor CCR8. CCR8 is one of the newer markers for tumor-resident Tregs, which is expressed on 30-80% of tumor-resident Tregs but minimally on other immune cells. CCR8 appears to be expressed on Tregs in response to antigen recognition, and reflects the enhanced immunosuppressive functions of the Tregs. Depleting CCR8+ Tregs leads to improved anti-tumor immunity without causing autoimmune complications, reinforcing the importance of tumor selectivity when achieving Treg depletion as an approach for cancer treatment.

### B. Discussion

The studies presented here indicate that tumor-specific targeting is a key when considering Treg depletion as a therapeutic approach in cancer management. However, it remains a challenge given the insufficient tumor specificity and cell type specificity of Treg-associated markers. CCR8 provides one of the few opportunities to achieve Treg depletion in a tumor-specific manner.

iRGD is a tumor-penetrating peptide (amino acid sequence: CRGDKGPDC (SEQ ID NO:1)) that delivers drugs deep into the extravascular tumor tissue in a tumor-specific manner. It carries a tumor-specific RGD motif that binds to αvβ3/β5 integrins and a tissue/cell-penetrating RXXK/R motif that binds to neuropilin-1 (NRP-1). Systemically injected iRGD homes to tumors by targeting the αvβ3/β5 integrins expressed on tumor endothelial cells. It is then proteolytically processed to expose an active RXXK/R motif that now binds to NRP-1. The RXXK/R-NRP-1 interaction activates a transcytotic penetration pathway in the tumor, which is mediated by vesicles that resemble macropinosomes. This mechanism allows iRGD as well as co-injected bystander molecules to penetrate through cell layers and widely spread into the tumor tissue.

Recent work has revealed that the expression of αvβ5 integrin in tumor tissue (in addition to NRP-1) is particularly important for iRGD to effectively penetrate the extravascular tumor tissue. As is reported herein, the αvβ5 integrin is also expressed on Tregs that infiltrate the PDAC tissue, but not those in the spleen, allowing iRGD to selectively target PDAC-resident Tregs. Accordingly, the αvβ5 integrin defines a subpopulation of CCR8⁺ Tregs that has potent immunosuppressive properties. In light of the findings herein, αvβ5 integrin is identified as a signature of highly immunosuppressive tumor-resident Tregs that has multiple implications for use in treating conditions where immune response need to be controlled such as graft-versus host disease. In addition, αvβ5 integrin is revealed to be a new target for inhibitors to be used in treating cancer such as PDAC including targeting by affinity probes such as the iRGD peptide.

Targeting CCR8 in mouse tumor models leads to tumor-specific depletion of Tregs, improved anti-tumor immunity, enhanced efficacy of ICBs, and most importantly, minimal autoimmune complications. Multiple clinical trials are underway to study the therapeutic significance of anti-CCR8 mAbs as monotherapy or in combination with an ICB or chemotherapy in solid tumor patients (e.g., NCT05537740, NCT05518045, NCT04895709). Our study identifies the αvβ5 integrin as another marker for tumor-resident Tregs, and expands the opportunity to selectively target the Tregs for improved PDAC therapy.

iRGD therapy in PDAC mice significantly increased the CD8/Treg ratio. The expansion of CD8⁺ T cells was likely a result of Treg depletion rather than direct CD8+ T cell targeting by iRGD because iRGD receptors were not detected on the CD8⁺ T cells. While the data suggest that iRGD reverses the immunosuppressive PDAC TIME, the effect was not sufficient to provide a meaningful anti-tumor effect because iRGD monotherapy did not inhibit tumor growth in line with previous studies. This may be due to the insufficient activation and/or exhaustion of the expanded CD8⁺ T cells given the high PD-L1 expression in our PDAC model. PD-1 expressed on CD8⁺ T cells inhibits naïve-to-effector differentiation of the cells and induces exhaustion of differentiated CD8+ T cells. Thus, blocking the PD-1/PD-L1 interaction is often a necessary step to gain enhanced and prolonged effector function of CD8+ T cells against infections and cancers. The finding that iRGD and PD-L1 blockade provided a synergistic therapeutic effect reflects this argument. It is also in line with data that show that the delivery of cytotoxic CD8⁺ T cells into PD-L1^{high} gastric tumors using iRGD only resulted in a moderate anti-tumor effect, but genetic deletion of PD-1 in the CD8+ T cells significantly enhanced the therapeutic efficacy.

Our finding that TCR stimulation was an inducer of αvβ5 integrin on Tregs suggests that antigen recognition is the key and that the αvβ5 integrin may serve as a marker for antigen-specific Tregs. This may explain why Tregs express the integrin in the presence of PDAC cells in vitro and in vivo. Historically, antigen-specific Tregs were believed to be iTregs derived from naïve CD4⁺ T cells, but recent studies have revealed that nTregs can also be a source if proper stimuli are provided. Indeed, our data show that αvβ5 integrin⁺ Tregs can be induced from naïve CD4⁺ T cells as iTregs, and also from nTregs that had already differentiated. The fact that resting nTregs prior to TCR stimulation lacked αvβ5 integrin expression suggests that the integrin is not a pan-Treg marker but manifests the activation status of Tregs, supporting the idea that it is a potential activation marker. Unlike CD25, a pan-T cell activation marker, the αvβ5 integrin appears to be tightly linked to Tregs given its abundant expression on CD4⁺ CD25⁺ Foxp3⁺ iTregs compared to the minimal expression state on CD4⁺ CD25⁺ Foxp3^{neg} T cells after TCR stimulation.

Antigen-specific Tregs are highly activated, and express various chemokines, cytokines, and their receptors. One of them is the chemokine receptor CCR8, which is expressed on immunosuppressive Tregs that have been activated upon TCR stimulation. CCR8⁺ Tregs are enriched in the TIME, and have become an important therapeutic target to achieve tumor-specific Treg depletion. It is remarkable that αvβ5 integrin⁺ Tregs were found to be a subpopulation of CCR8⁺ Tregs and that they were significantly more immunosuppressive than the αvβ5 integrin-negative counterpart. This strongly suggests that αvβ5 integrin⁺ Tregs are indeed the functionally dominant fraction of CCR8⁺ Tregs. The fact that the Tregs were highly effective against CD8⁺ T cells provides a plausible explanation to the significant expansion of CD8⁺ T cells in PDAC tissue noted upon Treg targeting achieved with iRGD. Notably, αvβ5 integrin expression appears to be restricted to Tregs more than CCR8, making it possible that αvβ5 provides an opportunity to further improve the tumor specificity of Treg targeted therapy.

Tregs are thought to suppress the proliferation and functions of T cells through direct contact with the cells and indirectly by modulating antigen presenting cells [REFs]. Given the role of αvβ5 integrin as an adhesion molecule, it is reasonable to speculate that the integrin enhances Treg engagement to the responder cells. αvβ5 integrin may also provide additional benefit by enhancing Treg survival considering its role in protecting cells from extrinsic apoptosis. Our data show that iRGD induces apoptosis of αvβ5 integrin⁺ Tregs in an RGD-dependent manner, indirectly suggesting that αvβ5-dependent survival signals are present in the Tregs and that blockage of the signals may contribute to iRGD-mediated depletion of tumor-resident Tregs.

In summary, this report conveys an important concept of tumor-specific Treg targeting, which can be achieved using an affinity probe that targets the αvβ5 integrin expressed on tumor-resident Tregs. Importantly, a recent study using our humanized PDAC (huPDAC) mice suggests that the concept may be translated to human PDAC patients. The immune system of huPDAC mice is replaced by functional human immune cells, which react to antigens in a human-leukocyte antigen (HLA)-restricted manner, allowing us to study the response of human immunity against human PDAC in mice. The huPDAC tumors were found to harbor αvβ5 integrin- and NRP-1-positive human Tregs, and treatment of the mice with iRGD monotherapy increased the CD8/Treg ratio in the tumors. Our finding that patient-derived PDAC tissue also harbors αvβ5 integrin- and NRP-1-positive Tregs further supports the argument that iRGD-mediated Treg targeting can be achieved in human PDAC patients. Clinical trials are now being organized to to explore the therapeutic benefit of adding an ICB to iRGD-based chemotherapy in PDAC patients.

### C. Embodiments

According to one embodiment, the invention relates to a method of treating cancer in a subject by administering to the subject an αvβ5 integrin inhibitor, and optionally, co-administering an iRGD peptide, or peptide variant thereof, or iRGD conjugate in combination with one or more immune checkpoint inhibitors. Examples of immune checkpoint inhibitors include but are not limited to a PD-1 inhibitor, a PD-L1 inhibitor, or a PD-L2 inhibitor or any combination thereof. The αvβ5 integrin inhibitor can include αvβ5 integrin specific antibodies or affinity probes (including aptamers), small molecule αvβ5 integrin inhibitors, or inhibitory oligonucleotides to reduce αvβ5 integrin expression.

According to another embodiment, the invention relates to a method of obtaining a highly immunosuppressive Treg population involving obtaining a sample of CD4+ cells from a subject and subjecting the CD4+ cells with one or more of anti-CD3 antibody, anti-CD28 antibody or anti-CD3/CD28 bispecific antibody and optionally TGFβ1 under conditions to produce a population of CD4+Foxp3+ Tregs and isolating from the population cells expressing αvβ5 integrin. The population of highly immunosuppressive Treg cells are CCR8+ cells that express αvβ5 integrin. Alternatively, the highly immunosuppressive cells can be obtained by first obtaining a tumor sample (e.g. a pancreatic tumor sample) from a subject and isolating cells expressing αvβ5 integrin from the tumor sample.

According to further embodiment, the invention relates to a method for reducing immune responses in a subject in need. The method involves administering to the subject a composition comprising αvβ5 integrin expressing CCR8+ Treg cells. In certain examples, the subject in need is a subject who has symptoms of an autoimmune disorder or is one who has received a tissue transplant. In a particular example, the subject in need is one who is experiencing graft-versus-host disease. In this example, the graft material may be subjected to the αvβ5 integrin expressing CCR8+ Treg cells before transplantation, or the subject is administered the cells following transplantation.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. Peptides

The peptides used in this study were synthesized in-house as previously described. In brief, they were synthesized with a Libery™ automatic microwave-assisted peptide synthesizer (CEM Corporation™) using standard solid-phase Fmoc/t-Bu chemistry. Some of the peptides were labeled with 5(6)-carboxyfluorescein (FAM) separated with a 6-aminohexanoic acid spacer.

### B. Tumor Cells

KPC-derived PDAC cells were established from the tumors of transgenic KrasG12D/+; LSLTrp53 R172H/+; Pdx-1-Cre mice as previously reported, and were authenticated using eighteen mouse short tandem repeat by ATCC (Manassas, VA). The cells were labeled with luciferase using a lentivirus that encoded the firefly luciferase cDNA linked to the neomycin resistant cDNA via a P2A cleavage peptide (LV-Fluc-P2A-Neo; Imanis Life Sciences™). The cells were cultured in Dulbecco's modified Eagle medium with 10% fetal bovine serum (FBS) and a penicillin-streptomycin mixture. The cells tested negative for mycoplasma contamination.

### C. Tumor Mouse Models

All animal experiments were performed according to procedures approved by the Institutional Animal Care and Use Committee (IACUC) at Columbia University (New York, NY) or the University of California San Diego (UCSD, La Jolla, CA).

Syngeneic PDAC mice were generated by orthotopic pancreatic injections of 5.0×105 KPC-derived PDAC cells into 8- to 10-week-old C57B6129SF1/J hybrid mice (Jackson™), which are the offspring of a cross between C57BL/6J females and 129S1/SvImJ males. In some experiments, the tumor growth was measured by luminescence imaging.

The mice were anesthetized with isoflurane, and the body hair was shaved. The mice received intraperitoneal (IP) injection of 2 milligrams of luciferin (Promega™ Corporation) and subjected to luminescence imaging using an IVIS® Spectrum In Vivo Imaging System (PerkinElmer™, Inc). Transgenic KPC mice were maintained as described previously. Healthy C57BL/6 mice were used to isolate T cells for immunosuppression assays as described herein.

### D. Human Sample Collection

Human experiments were performed according to procedures approved by the Institutional Review Board at Columbia University (protocol #AAAT1231). Anonymized human specimens from scheduled operations were obtained when the pathologist determined that excess tissue was available for research purposes.

### E. Mouse Treatment Studies

Long-term treatment studies using combinations of iRGD, Gem, and Nab-P in transgenic KPC mice and subsequent immunohistochemistry (IHC) were performed as previously reported.

The treatment studies in orthotopic syngeneic PDAC mice were performed as follows. Eight days after orthotopic implantation of 5.0×105 KPC-derived PDAC cells, the mice were randomized into respective treatment cohorts, such as intravenous (IV) phosphate buffered saline (PBS) alone, IV iRGD alone (300 μg/25 g), IV PBS+IP antimouse PD-L1 mAb (200 μg/mouse; clone MIH6; BioLegend™), IV iRGD+IP anti-mouse PD-L1 mAb, IV PBS+IV Gem (4 mg/kg; MilliporeSigma™), and IV iRGD+IV GEM+IP anti-mouse PD-L1 mAb. The treatment was given 3 times a week for 2 weeks starting 8 days after tumor cell implantation. The tumors and major organs were harvested at the end of the studies on day 22, weighed, and processed for flow cytometry, IF, and IHC. The treatment studies were terminated according to the guidelines by the IACUC at Columbia University. Treatment studies in transgenic KPC mice were performed as previously described.

F. Isolation of Immune Cells from PDAC Tissue

Tumors from orthotopic PDAC mice were minced into 2-4 mm pieces and added with an enzyme mix from a tumor dissociation kit (Miltenyi Biotec™) in Roswell Park Memorial Institute medium 1640 (Cytiva™). The tissues were dissociated using a gentle MACS Dissociator (Miltenyi Biotec™) followed by a 40-minute incubation at 37° C. The samples were then centrifuged for 10 minutes at 3000×g in the presence of debris removal solution (Miltenyi Biotec™) and 10 minutes at 1000×g in cooled PBS. The supernatant was aspirated to remove the debris. Splenocytes were prepared by gently grinding the spleen and filtrating the resulting cell suspension through a 40 μm cell strainer. Red cells were lysed with an ACK lysing buffer (Thermo Fisher Scientific™

The isolated immune cells were subjected to flow cytometry for quality check and to analyze the cell populations of interest. To test FAM-iRGD binding, the isolated cells were incubated with 10 μM FAM-iRGD for 1 hour at 37° C. in a binding buffer containing 1.0% bovine serum albumin (BSA), 150 mM sodium, 1.0 mM magnesium, and 1.0 mM calcium. The binding was analyzed by flow cytometry.

### G. In Vitro Co-Culture System of CD4+ T Cells and KPC-Derived PDAC Cells

CD4+ T cells were magnetically isolated from the spleens of healthy C57B6129SF1/J mice using a mouse CD4+ T cell isolation kit (Miltenyi Biotec™). The CD4+ T cells were cultured for 3 days with or without KPC-derived PDAC cells in the presence of low dose anti-CD3/CD28 beads (×40 dilution; Miltenyi Biotec™), TGF-β1 (5 ng/ml; Bio-Techne Corporation™), and recombinant mouse interleukin-2 (mIL-2, 100 U/ml; Roche™), and subjected to flow cytometry as described elsewhere. In some cases, 1.0 mM iRGD was added to the culture in binding buffer supplemented with mIL-2 to study the effect on apoptosis. The cells were then stained with FITC-conjugated annexin V (BioLegend™) and 7-AAD (BioLegend™) for 15 minutes at room temperature to detect apoptotic cells by flow cytometry.

### H. In Vivo Peptide Homing Assay

C57B6129SF1/J mice bearing 22 day-old orthotopic KPC tumors received an IV bolus of PBS or FAM-iRGD (300 μg/25 g). One hour later, the mice were perfused through the heart with PBS under deep anesthesia, and the tumors were collected. The tissues were stained for Foxp3 and DAPI using Abs described elsewhere, and subjected to confocal microscopy.

### I. Flow Cytometry

Immune cells isolated from the tumor or spleen, or cells cultured in vitro were tested for surface marker by staining with the following reagents for 20 minutes at 4° C.: Brilliant Violet 650™-conjugated anti-mouse CD3 Ab (clone 17A2; BioLegend), PerCP/Cyanine5.5-conjugated anti-mouse CD4 Ab (clone GK1.5; BioLegend™), PE/Cyanine7-conjugated antimouse CD8a Ab (clone 53-6.7; BioLegend™), PE-conjugated anti-mouse CD25 Ab (clone 7D4; Miltenyi Biotec™), PE/Cyanine7-conjugated anti-mouse CD25 Ab (clone PC61; BioLegend™), Brilliant Violet 421™ or 711™-conjugated anti-mouse CD304/NRP-1 Ab (clone 3E12; BioLegend™), Alexa Fluor® 647-conjugated anti-mouse integrin αvβ5 Ab (clone ALULA; BD Biosciences™), PE-conjugated anti-mouse CD198 (CCR8) Ab (clone SA214G2; BioLegend™), Zombie NIR™ (BioLegend™), or Aqua™ Fixable Viability Kit (BioLegend™). Intracellular staining of immune cells was performed using the following Abs and a Foxp3 staining buffer kit (eBioscience) according to the manufacturer's instructions: Brilliant Violet 421™-conjugated anti-Foxp3 Ab (FJK-16s; eBioscience™) Pacific Blue™-conjugated anti-mouse Foxp3 Ab (clone MF-14; BioLegend™), cleaved caspase-3 (Asp175) (5A1E) rabbit mAb (Cell Signaling™), and Alexa Flour™ 488-conjugated donkey anti-rabbit secondary Ab (Thermo Fisher Scientific™)

Tregs were defined as CD4+CD25+ or CD4+Foxp3+ depending on the experimental design. PDL1 expression on KPC cells was assessed with an allophycocyanin-conjugated anti-mouse PD-L1 Ab (clone B7-H1; BioLegend™). Flow cytometry was performed using a Cytek Aurora™ (Cytek™) or BD LSRFortesa™ (BD Biosciences™), and the data were analyzed with FCS Express version 7.06.0015 (De Novo Software™) or FlowJo™ v10.8 (BD Biosciences™)

### J. Immunofluorescence

Mouse and human samples were fixed with 4% paraformaldehyde overnight, washed with PBS three times, and transferred to 30% sucrose at 4° C. until the tissues sank. Then, the tissue samples were embedded in optimal cutting temperature (OCT) compound to prepare m frozen sections. Paraffin-embedded 4 μm sections of human tissue were obtained through the Molecular Pathology/MPSR Core. The sections were stained with anti-CD3 Ab (clone CD3-12; Abcam™), anti-mouse CD4 Ab (clone GK1.5; Invitrogen™), fluorescein-conjugated anti-mouse CD8 Ab (clone YTS 105.18; Absolute Antibody™), anti-human/mouse Foxp3 Ab (clone 1054c; Novus Biologicals™), anti-mouse NRP-1 Ab (R&D Systems™ Inc.), antihuman NRP-1 Ab (clone AD5-17F6; Miltenyi Biotec™), Alexa Fluor® 647-conjugated antimouse integrin αvβ5 Ab (clone ALULA; BD Biosciences™), Ultra-LEAF™ purified antimouse PD-L1 Ab (clone 10F.9G2; BioLegend™), 4′,6-diamidino-2-phenylindole (DAPI; Thermo Fisher Scientific™) followed by an appropriate secondary Ab with Alexa Fluor 488, 546 or 680 (Thermo Fisher Scientific™)

Images were taken with an LSM 710 confocal microscope system (Zeiss™). A ZEN 3.0 SR black edition software (Zeiss™) was used for acquisition and analysis of the images. In some experiments, cells of interest were counted in five randomly chosen high-power fields (HPFs) per section to obtain an averaged number. The cell count was performed for three or four mice per group as indicated in each experiment.

### K. Statistical Analyses

The Mann-Whitney U test was used to compare two groups with four or more samples per group. The Welch's test was used to compare two groups with three samples per group. One-way analysis of variance (ANOVA) was used to compare three or more groups with a normal distribution. One sample Wilcoxon signed rank test was used when the data was not assumed to be normally distributed in each group. All statistics were performed using GraphPad Prism™ (Ver. 8.4.3).

### Example 2: In Vitro Induction of αvβ5 Integrin+ Tregs from Naïve CD4+ T Cells and nTregs

Naïve CD4⁺ T cells and nTregs were isolated from the spleen of healthy C57B6129SF1/J mice by magnetic separation using a mouse CD4+ T cell isolation kit and PE-conjugated anti-mouse CD25 with anti-PE magnetic separation micro beads (Miltenyi Biotec™). The isolated T cells were stimulated with anti-CD3/CD28 beads (×25 dilution; Miltenyi Biotec™) in the presence or absence of recombinant mouse TGF-β1 (5 ng/ml; Bio-Techne™ Corporation) for 3 days. iRGD (100 μM) or iRGE (100 μM) peptide was added to the cultures in some experiments. To study the effect of TGF-β1 on the induction of αvβ5 integrin+ iTregs, naïve CD4+ T cells were cultured with anti-CD3/CD28 beads (x 25 dilution) and TGF-β1 (5 ng/ml) in the presence or absence of varying concentrations of a TGF-βR1 inhibitor (LY2157299; Selleck™ Chemicals LLC). To study the effect of TCR stimulation on the induction of αvβ5 integrin+ iTregs, naïve CD4+ T cells treated with varying concentrations of plate-coated anti-mouse CD3 Ab (eBioscience™) in the presence of soluble anti-mouse CD28 Ab (2 μg/ml) (eBioscience™) and TGF-β1 (5 ng/ml). The cells were subjected to flow cytometry as described elsewhere.

### Example 3: In Vitro Treg Suppression Assay

Naïve CD4+ T cells isolated from the spleen of C57B6129SF1/J or C57BL/6 mice were treated with anti-CD3/CD28 beads and TGF-β1 for 3 days as described above. mIL-2 (10 ng/ml; R&D Systems™, Inc.) was added to the culture medium from day 1 to expand iTregs. The resulting cell suspension was treated with a PE-conjugated anti-CD25 Ab (Miltenyi Biotec), PE-conjugated anti-CCR8 Ab (eBioscience), and/or Alexa Fluor® 647-conjugated anti-β5 integrin Ab (clone ALULA; BD Biosciences) combined with an anti-PE or -AF647 magnetic separation technique (Miltenyi Biotec™) to enrich for CCR8+ iTregs, αvβ5 integrin+ CCR8+ iTregs, or αvβ5 integrinneg CCR8+ iTregs. Responder CD4+CD25neg T cells and CD8+ T cells (Tconv) were isolated from the spleen of healthy C57B6129SF1/J or C57BL/6 mice by magnetic separation and labeled with CellTrace™ Violet (CTV; Thermo Fisher Scientific™). The Tconv and iTregs were mixed in different ratios and co-cultured in the presence of anti-CD3/CD28 beads (×25 dilution). Division of the Tconv was assessed by measuring the dilution of CTV by flow cytometry on day 3.

### Example 4: Long-Term iRGD Therapy Increases CD8+ T Cells in the PDAC Tissue in Transgenic KPC Mice

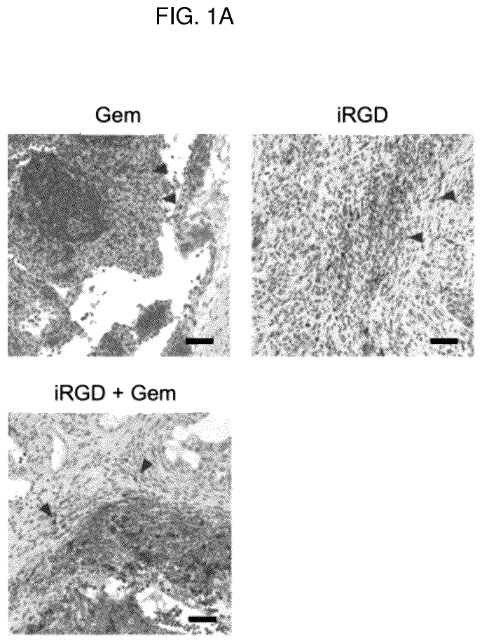

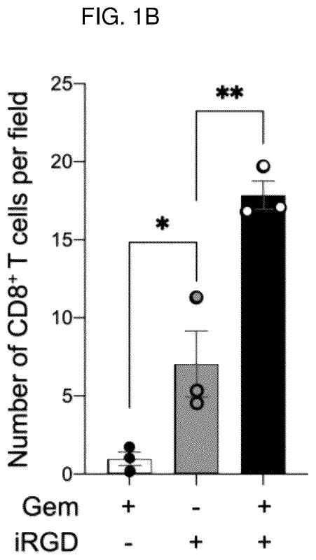

For the data presented in FIG. 1, primary PDAC tumors were collected from transgenic KPC mice that received long-term treatment with Gem, iRGD, or iRGD+Gem as part of a previously published survival study. The primary PDAC tumors were immunohistochemically analyzed for the presence of CD8+ T cells.

FIG. 1A shows representative images of CD8+ T cells in the PDAC tissue (brown arrowheads show examples). Scale bars, 50 μm. FIG. 1B is a bar diagram showing the number of CD8+ T cells per high-power field (n=3) per arm. The statistical analysis was one-way ANOVA; p=0.0468 (Gem vs iRGD), p=0.0034 (iRGD vs Gem+iRGD). The error bars indicate the mean±standard error; *p<0.05; **p<0.01.

We have previously reported that iRGD+Gem significantly prolonged the survival of transgenic KrasG12D/+; LSL-Trp53R172H/+; Pdx-1-Cre (KPC) PDAC mice compared to iRGD or Gem therapy alone. Further analysis of the study revealed that PDAC samples from the iRGD+Gem arm harbored significantly more CD8⁺ T cells than those from the Gem monotherapy arm (see FIG. 1).

### Example 5: Long-Term iRGD Therapy Decreases Tregs in the PDAC Tissue in Transgenic KPC Mice

Primary PDAC tumors were collected from transgenic KPC mice that received long-term treatment with or without Gem, iRGD, or iRGD+Gem as part of a previously published survival study. The presence of Foxp3+ Tregs in the primary PDAC tumors was analyzed by immunofluorescence.

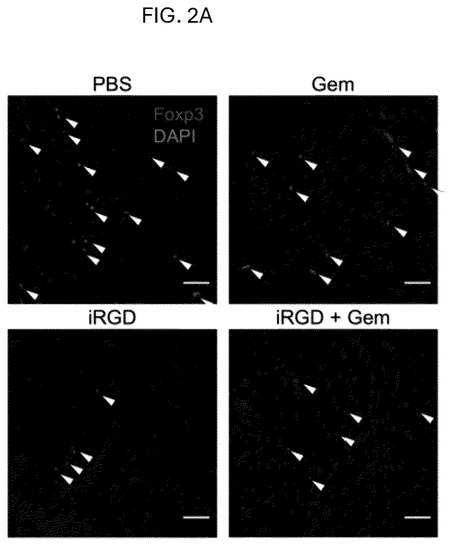

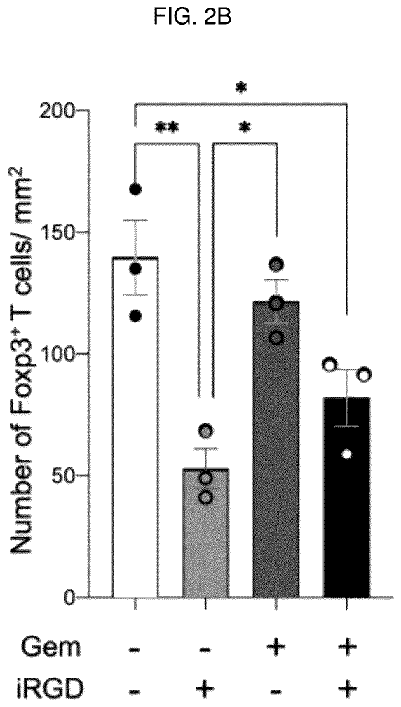

FIG. 2A shows representative images of Foxp3+ Tregs (red) in PDAC tissue. Blue, DAPI; scale bars, 25 μm. FIG. 2B is a bar diagram showing the number of Foxp3+ Tregs per high-power field in the primary PDAC tumors (n=3 per arm). The statistical analysis used was one-way ANOVA; p=0.0025 (PBS vs iRGD), p=0.0101 (iRGD vs Gem), p=0.0261 (PBS vs Gem+iRGD). The error bars indicate the mean±standard error; *p<0.05; **p<0.01.

Although less effective than iRGD+Gem, iRGD monotherapy also increased the number of CD8+ T cells in the PDAC tissue, suggesting that iRGD has an immunomodulatory effect. The hypothesis that the effect was a response to Treg targeting enabled by the iRGD peptide given that mouse Tregs express NRP-1, one of the iRGD receptors, appeared reasonable because there were less PDAC-resident cells expressing Foxp3, a master regulator of Tregs, in the iRGD monotherapy and iRGD+Gem combination therapy arms compared to the others (see FIG. 2).

### Example 6: Rapid Growth of Orthotopic KPC-Derived PDAC Tumors in B6129SF1/J Hybrid Mice

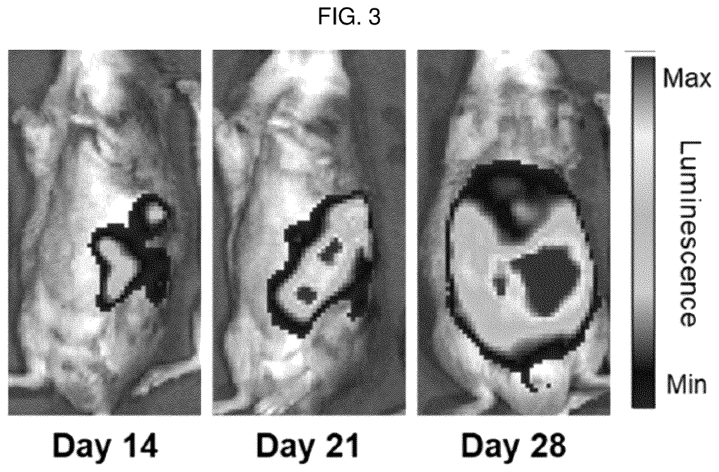

A syngeneic PDAC mouse model was generated using luciferase⁺ PDAC cells derived from KPC mice. The PDAC tumors rapidly grew orthotopically in B6129SF1/J hybrid mice (see FIG. 3). FIG. 3 shows representative images showing longitudinal growth of an orthotopic luciferase+ KPC-derived PDAC tumor in a mouse with a matching genetic background analyzed by bioluminescence imaging.

### Example 7: The Proportion of Tregs and CD8+ T Cells and the CD8/Treg Ratio in the Spleen of Normal Mice and PDAC Mice

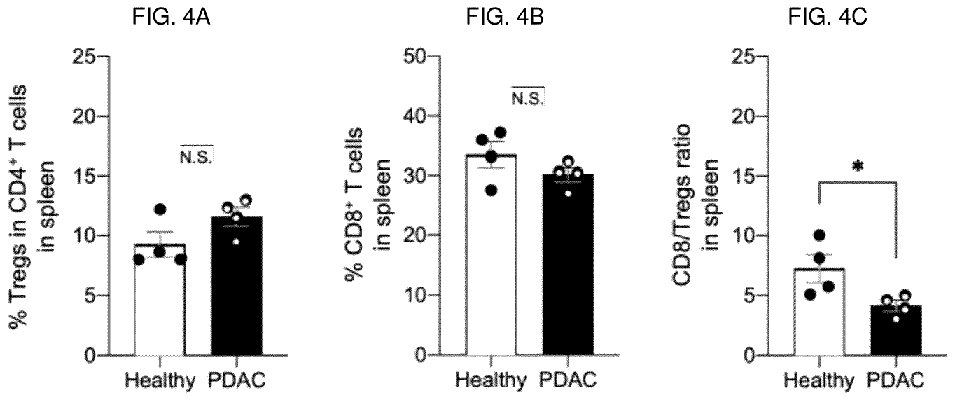

C57B6129SF1/J hybrid mice bearing orthotopic KPC-derived PDAC tumors were treated with PBS 3 times a week for 2 weeks. The spleen was collected 14 days after the treatment was started. FIG. 4A and FIG. 4B show the results. The proportion of CD4+CD25+ Tregs (FIG. 4A) and CD8+ T cells (FIG. 4B) in the spleen of healthy and PDAC mice were analyzed by flow cytometry. FIG. 4C shows data on the CD8/Treg ratio, calculated based on the values from FIG. 4A and FIG. 4B (n=4 per group). The statistical analysis used was the Mann-Whitney U test; p=0.1143 (FIG. 4A), p=0.2000 (FIG. 4B), p=0.0286. The error bars indicate the mean±standard error; *p<0.05; N.S., not significant.

The presence of the tumor had no statistically significant effect on the proportion of Tregs (see FIG. 4A) or CD8+ T cells (see FIG. 4B) in the spleen. However, the ratio of CD8+ T cells against Tregs (CD8/Treg ratio) in the spleen was slightly decreased when PDAC was present (see FIG. 4C).

### Example 8: IRGD Monotherapy does not Affect PDAC Growth

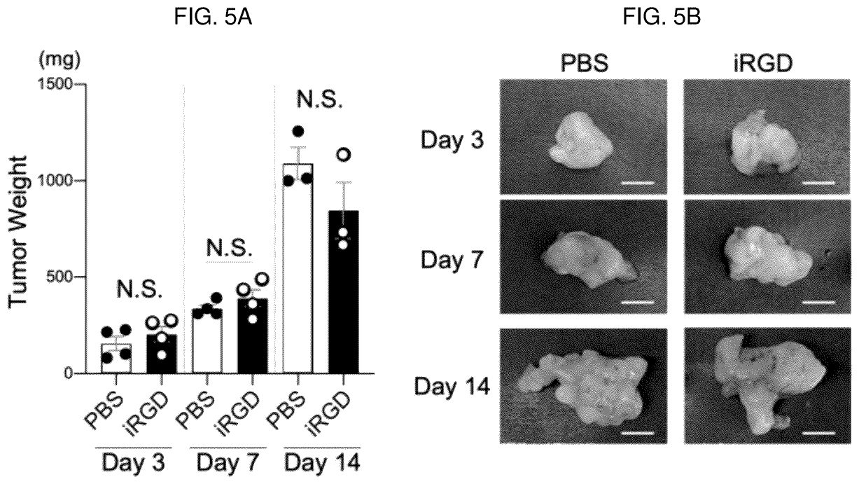

In line with previous studies, iRGD monotherapy did not affect tumor growth (see FIG. 5). C57B6129SF1/J hybrid mice bearing orthotopic KPC-derived PDAC tumors were treated with PBS or iRGD 3 times a week for 2 weeks. The tumors were collected and weighed 3, 7 and 14 days after the treatment was started. The results are summarized in the bar diagram of FIG. 5A (n=4 (day 3 and day 7); n=3 (day 14)). The statistical analysis used was the Mann-Whitney U test (day 3 and day 7) or Welch's t test (day 14); p=0.4857 (day 3), p=0.4857 (day 7), p=0.2389 (day 14). The error bars indicate the mean standard error; N.S., not significant. Representative images of the tumors are shown to the right in FIG. 5B. Scale bars, 5 mm.

### Example 9: IRGD Depletes Tregs, Expands CD8+ T Cells in PDAC Tissue, and Enhances the Efficacy of an Anti-PD-L1 Blockade

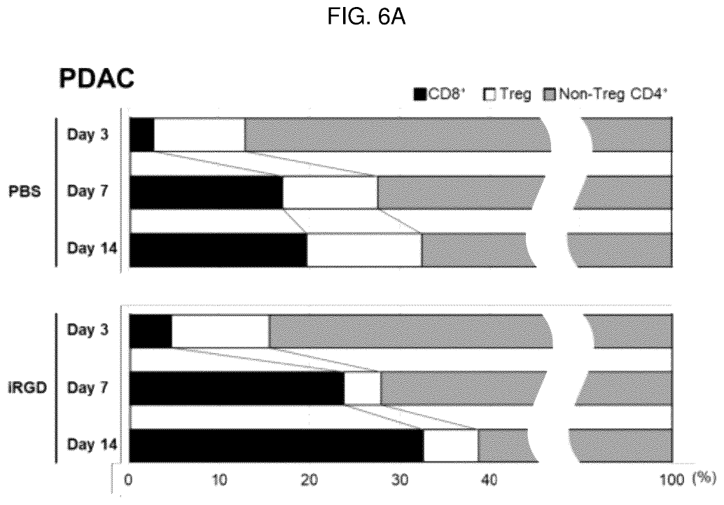

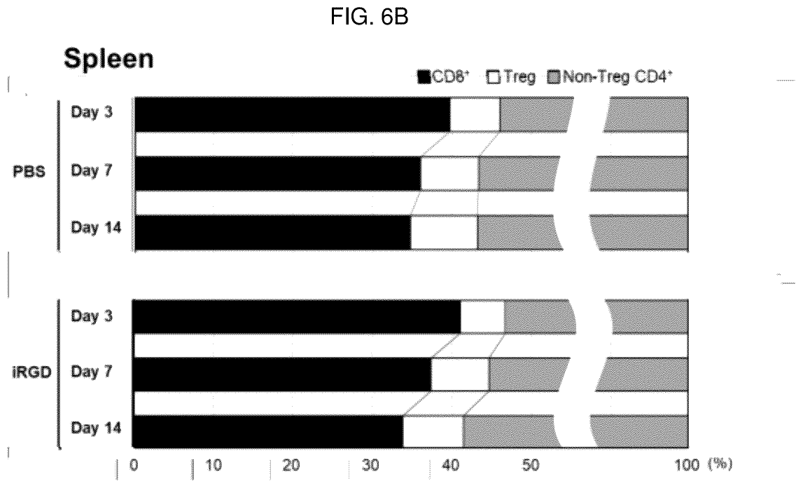

The Effect of iRGD Therapy on the Proportion of CD8+ T cells, Tregs, and Non-Treg CD4+ T Cells in the PDAC Tissue and the Spleen was tested. C57B6129SF1/J hybrid mice bearing orthotopic KPCderived PDAC tumors were treated with PBS or iRGD 3 times a week for 2 weeks. The tumors and spleen were collected 3, 7 and 14 days after the treatment was started. The proportion of CD8+ T cells, CD4+CD25+ T cells (Tregs), and CD4+CD25neg T cells (non-Tregs) in the tumors (FIG. 6A) and the spleen (FIG. 6B) were analyzed by flow cytometry (n=3 per arm).

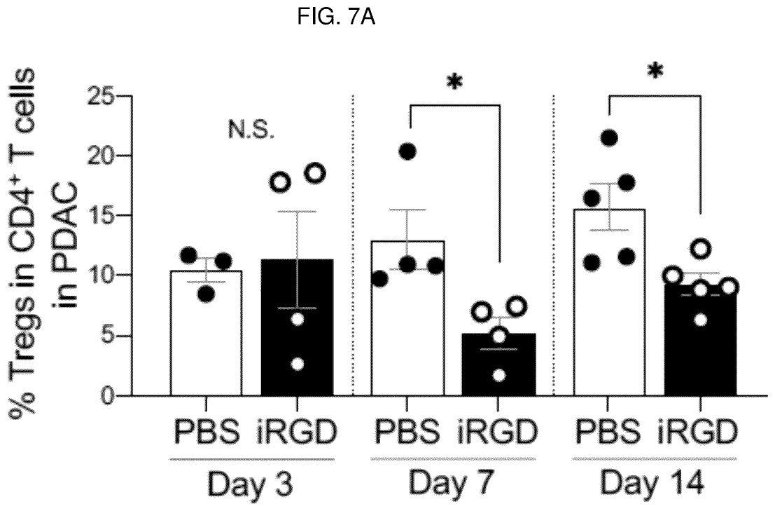

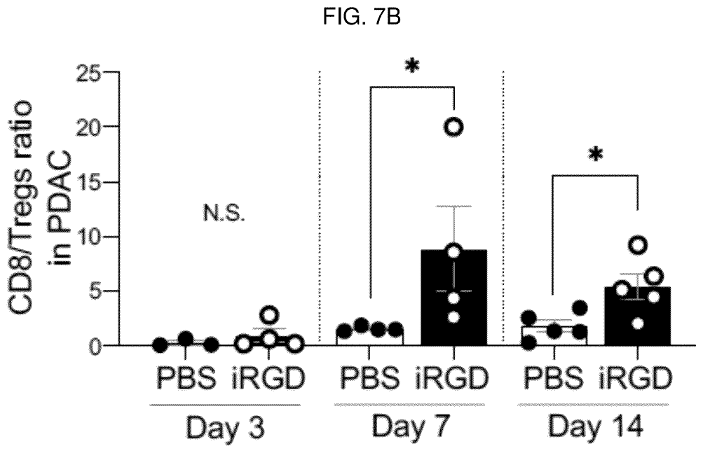

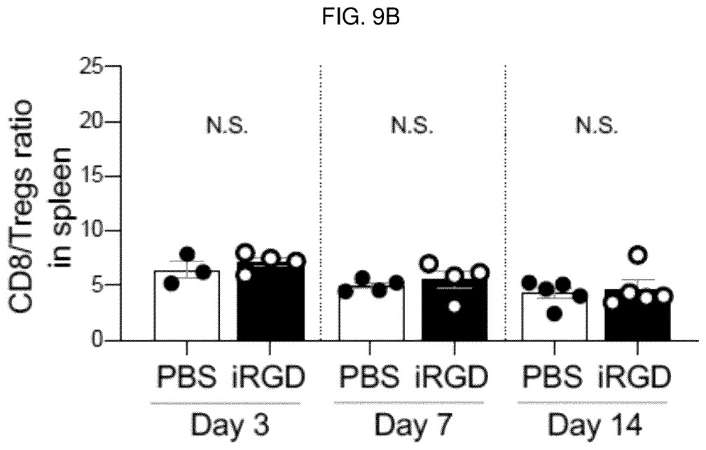

In addition, C57B6129SF1/J hybrid mice bearing KPC-derived orthotopic PDAC tumors were intravenously treated with PBS or iRGD 3 times a week for 2 weeks. Tumors and spleen were collected at 3, 7 and 14 days after the treatment was started. The proportion of CD4+CD25+ Tregs among CD4+ T cells in the tumor (see FIG. 7A, left panel) or the spleen (see FIG. 9A, left panel), and the ratio of CD8+ T cells over Tregs (CD8/Treg ratio) in the tumor (see FIG. 7B, right panel) or the spleen (see FIG. 9B, right panel) were analyzed by flow cytometry (n=3 (day 3, PBS), 4 (day 3, iRGD; day 7) or 5 (day 14) per group).

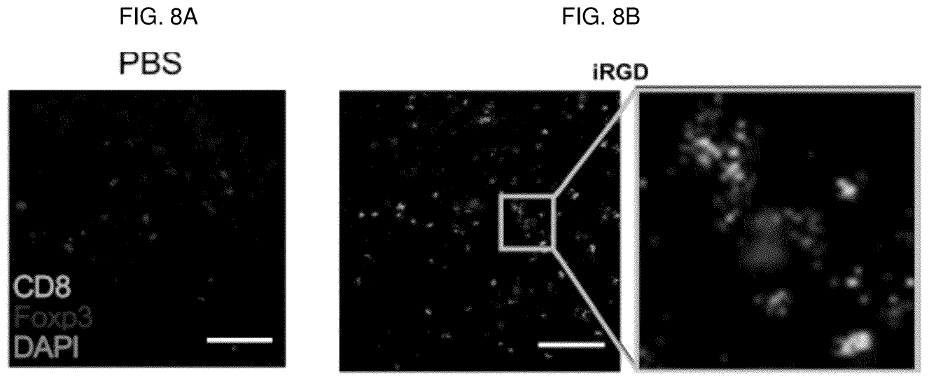

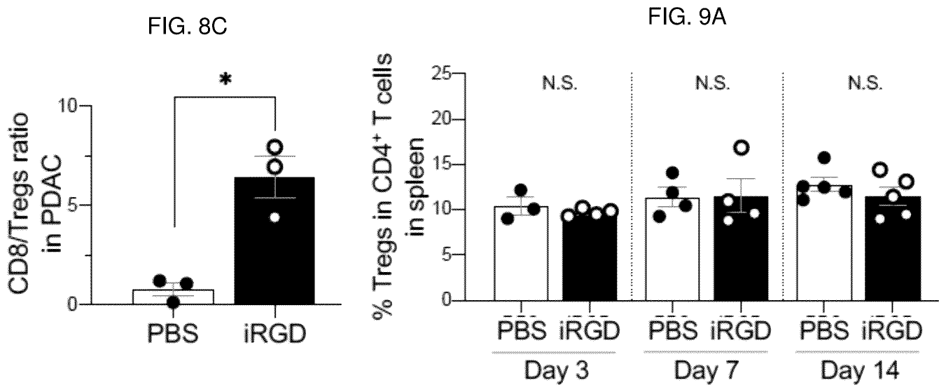

Representative images of IF performed on the PDAC tissue harvested after 14 days of treatment are shown in FIG. 8A and FIG. 8B (green, CD8+ T cells; red, Foxp3+ Tregs; blue, DAPI; scale bars, 50 μm). The bar diagram in FIG. 8C shows the CD8/Treg ratio, which was measured by counting the cells in 5 random high-power fields per section for 3 mice per group.

Statistical analysis was as follows: Welch's t test (for n=3) or Mann-Whitney U test (for n≥4); p=0.8454 (FIG. 7A, left, day 3), p=0.0286 (a, left, day 7), p=0.0317 (FIG. 7A, left, day 14), p=0.3656 (FIG. 7B, right, day 3), p=0.0286 (FIG. 7B, right, day 7), p=0.0317 (FIG. 7B, right, day 14), p=0.0243 (FIG. 8A and FIG. 8B), p=0.5195 (FIG. 9A, left, day 3), p=0.8857 (FIG. 7A, left, day 7), p=0.5476 (FIG. 9A, left, day 14), p=0.4696 (FIG. 9B, right, day 3), p=0.3429 (FIG. 9B, right, day 7), p=0.6905 (FIG. 9B, right, day 14).

In summary, flow cytometry of tumor-resident immune cells revealed that iRGD significantly reduced the proportion of Tregs (FIG. 7A, left panel), and increased the proportion of CD8+ T cells (FIG. 6A) and the CD8/Treg ratio in the PDAC tissue in 7 days (FIG. 7B, right panel). Similar results were obtained by immunofluorescence (IF) (FIG. 8A and FIG. 8B). In contrast, iRGD monotherapy did not affect the proportion of Tregs or CD8+ T cells or the CD8/Treg ratio in the spleen of the PDAC mice (FIG. 9 and FIG. 6B), suggesting that the effect of iRGD on the immune cells was tumor specific.

### Example 10: IRGD in Combination with an ICB Reduces Tumor Growth

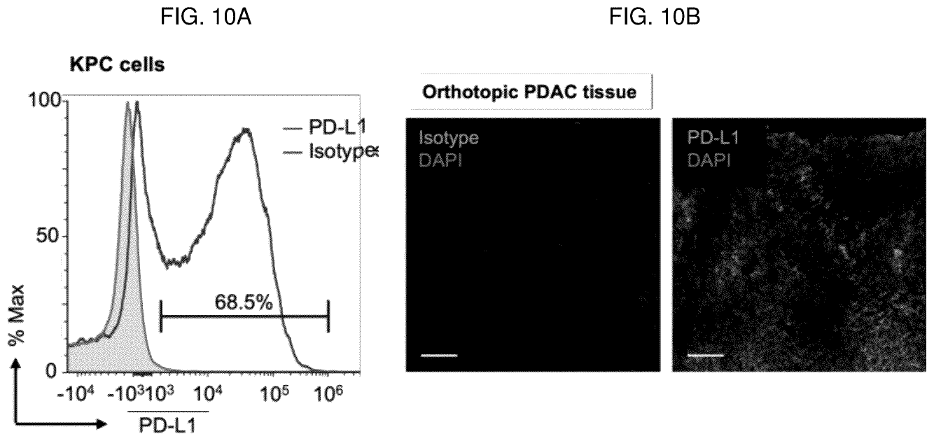

The ability of iRGD to reduce PDAC-resident Tregs prompted the hypothesis that iRGD could improve the efficacy of ICBs against PDAC. We elected to use a monoclonal Ab (mAb) against programmed cell death ligand-1 (PD-L1) given the high expression of PD-L1 in our orthotopic PDAC model (FIG. 10).

PD-L1 expression on KPC derived PDAC cells analyzed by flow cytometry. See FIG. 10A (blue, isotype control; red, anti-PD-L1 Ab). FIG. 10B shows representative immunofluorescence images showing the expression of PD-L1 (green) in orthotopic KPC-derived PDAC tissue that was harvested 22 days after tumor cell inoculation (blue, DAPI; scale bars, 50 μm).

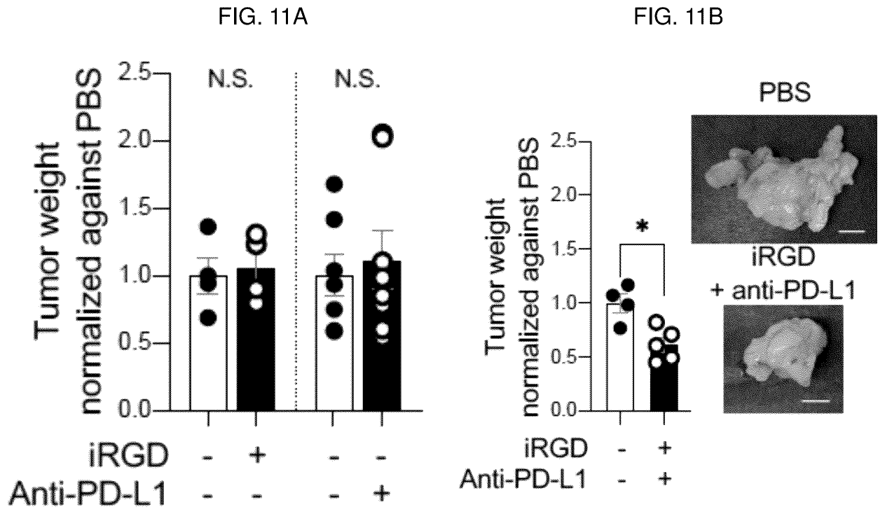

Therefore, KPC-derived orthotopic PDAC mice were treated with PBS, iRGD, anti-PD-L1 Ab, or iRGD+anti-PDL1 mAb (see FIG. 11A and FIG. 11B) or iRGD+anti-PD-L1 mAb with or without Gem (see FIG. 11C) 3 times a week for 2 weeks. The weight of the tumors at the end of the study are shown. Representative tumor images are shown to the right.

While iRGD or the anti-PD-L1 mAb alone did not affect tumor growth (FIG. 11A), iRGD in combination with the anti-PD-L1 mAb led to a significant decrease in the tumor volume (FIG. 11B). Adding the anti-PD-L1 mAb to the iRGD+Gem regimen also enhanced the anti-tumor effect (FIG. 11C).

Scale bars, 5 mm. n=4, 7 or 8 per arm (FIG. 11A); n=4 or 5 per arm (FIG. 11B); n=11 per arm (FIG. 11C). Statistical analysis, Mann-Whitney U test; p>0.9999 (FIG. 11A, left), p=0.7789 (FIG. 11A, right), p=0.0317 (FIG. 11), p=0.0192 (FIG. 11C). Error bars, mean±standard error; *p<0.05; N.S., not significant.

### Example 11: PDAC-Resident Tregs Express the αvβ5 Integrin

The expression of av integrins along with NRP-1 is essential for iRGD to target cells properly. Therefore we investigated whether PDAC-resident Tregs express av integrins in addition to the mouse Treg marker NRP-128, allowing iRGD to effectively target them in the PDAC tissue.

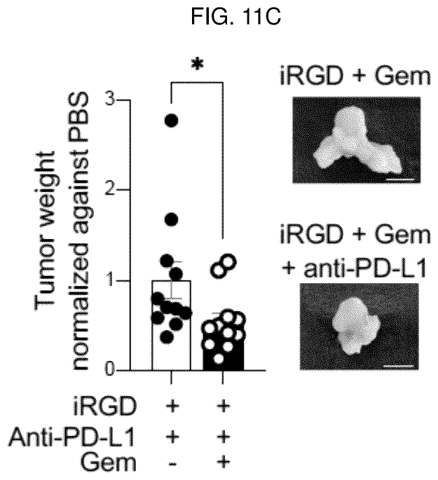

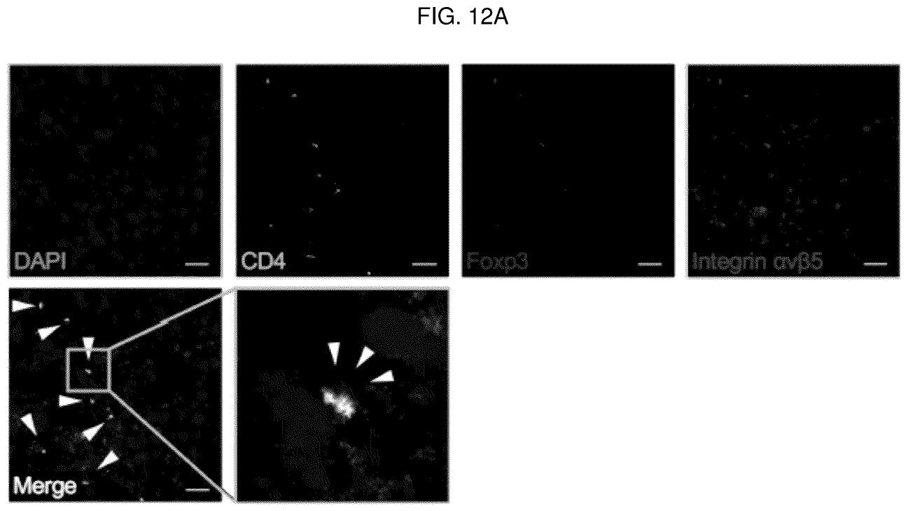

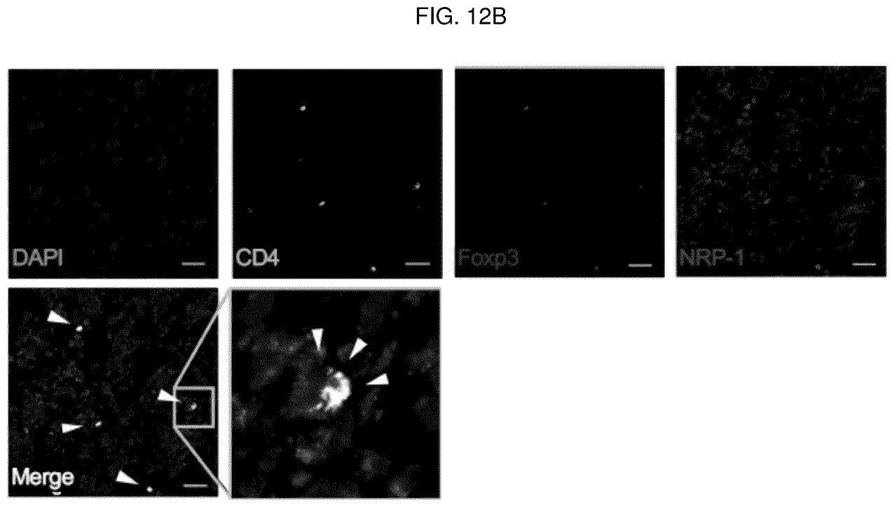

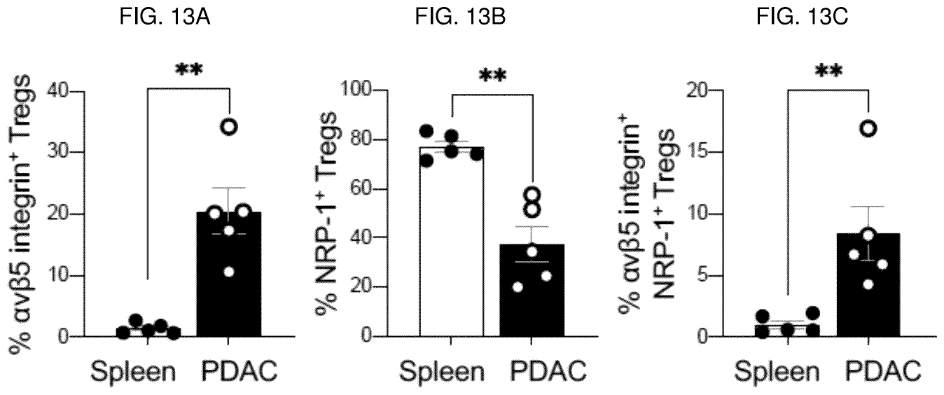

For the data in FIG. 12A and FIG. 12B, representative confocal images of αvβ5 integrin+CD4+Foxp3+ T cells (FIG. 12A) and NRP-1+CD4+Foxp3+ T cells (FIG. 12B) in the PDAC tissue of KPC-derived orthotopic PDAC mice. Arrows indicate αvβ5 integrin+(FIG. 12A, magenta) or NRP-1+(FIG. 12B, magenta) Tregs. Green, CD4; red, Foxp3; blue, DAPI. The boxed areas are magnified. Scale bars, 20 μm. For the data in FIG. 13, flow cytometry analysis showed the proportion of CD4+CD25+ Tregs positive for αvβ5 integrin, NRP-1, or both in the spleen and PDAC in KPC derived orthotopic PDAC mice (n=5 per group).

IF showed that αvβ5 integrin+ and NRP-1+ Tregs were indeed present in the tumor tissue of KPC-derived orthotopic PDAC mice (FIG. 12A and FIG. 12B). Flow cytometry showed that the αvβ5 integrin was expressed on approximately 20% of the Tregs (FIG. 13). Interestingly, αvβ5 integrin was rarely expressed on splenic Tregs suggesting that selective expression of the αvβ5 integrin on PDAC-resident Tregs allowed iRGD to induce tumor-specific Treg reduction.

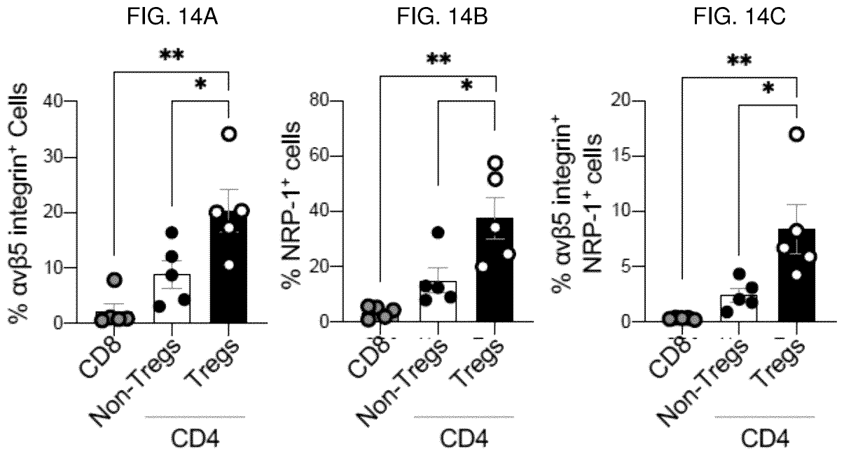

See FIG. 14 for flow cytometry analysis showing the proportion of CD8+ T cells, CD4+CD25neg T cells (non-Tregs), and CD4+CD25+ Tregs that are positive for αvβ5 integrin, NRP-1, or both in the PDAC of KPC-derived orthotopic PDAC mice (n=5 per group). As expected, NRP-1 was expressed on both PDAC and splenic Tregs. The expression of αvβ5 integrin and NRP-1 was significantly lower on other CD4+ T cells and almost non-detectable on CD8+ T cells (FIG. 14).

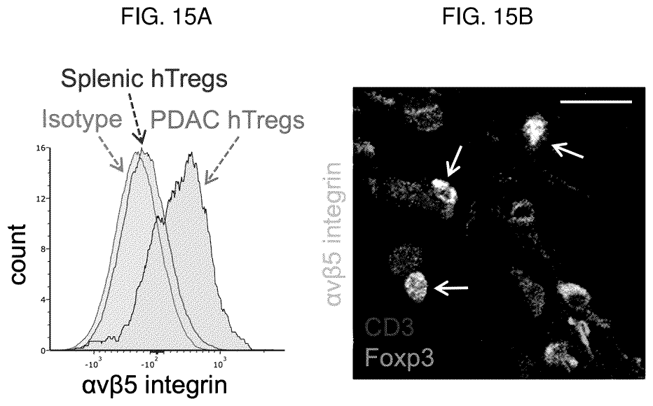

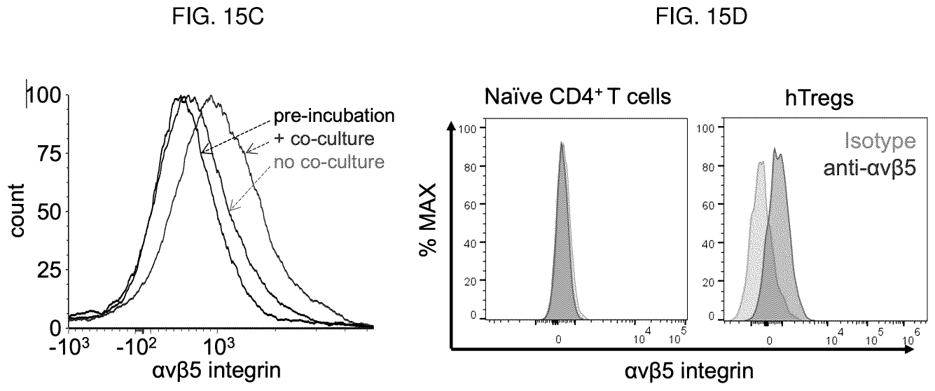

FIG. 15 shows that αvβ5 integrin+ Tregs are found in human PDAC tissue. Flow cytometry of PDAC and spleen tissue derived from human patients shows that human Tregs (hTregs) in the PDAC express the αvβ5 integrin but hTregs in the spleen from the same patient does not (FIG. 15A). FIG. 15B presents a representative confocal image of αvβ5 integrin+Foxp3+CD3+ Tregs in human PDAC. Arrows show examples. αvβ5 integrin+ hTregs can be induced from human splenocytes by co-culturing them with PDAC cells isolated from the sample patient (FIG. 15C) or by providing TCR and TGF-β stimulation in vitro (FIG. 15D).

In FIG. 12 through FIG. 15, color, αvβ5 integrin (left) or NRP-1 (right); color, CD3; color, Foxp3. Scale bars, 20 μm. The bar diagrams summarize the proportion of αvβ5 integrin+ and NRP-1+ Tregs. n=X. For the statistical analysis, Mann-Whitney U test (FIG. 13) or one-way ANOVA (FIG. 14); p=0.0079 (FIG. 13A, left), p=0.0079 (FIG. 13B, middle), p=0.0079 (FIG. 2C-13C, right), p=0.0015 (FIG. 2D-14A, left, CD8 vs Tregs), p=0.0296 (FIG. 14A, left, Non-Tregs vs Tregs), p=0.0012 (FIG. 14B, middle, CD8 vs Tregs), p=0.0195 (FIG. 14B, middle, Non-Tregs vs Tregs), p=0.0028 (FIG. 14C, right, CD8 vs Tregs), p=0.0203 (FIG. 14C, right, Non-Tregs vs Tregs). Error bars indicate the mean standard error (*p<0.05; **p<0.01).

### Example 12: IRGD Binds to αvβ5 Integrin+ Tregs Induced in the Presence of PDAC Cells

The results above suggest that the αvβ5 integrin is a tumor-specific marker for PDAC-resident Tregs, and that the TME is critical for the induction of αvβ5 integrin+ Tregs. We therefore hypothesized that PDAC cells help induce αvβ5 integrin+ Tregs. To test this hypothesis, we magnetically isolated CD4+ T cells from mouse splenocytes and cultured the cells on a monolayer of KPC-derived PDAC cells for 3 days.

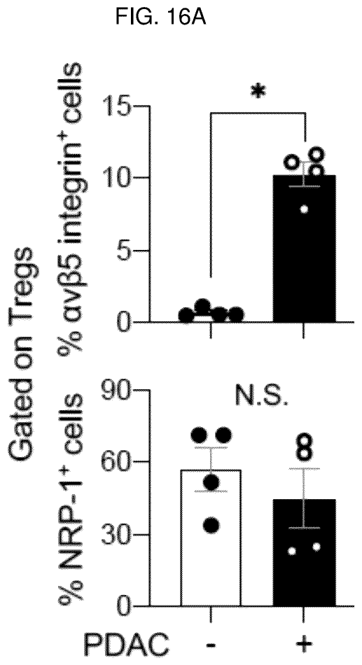

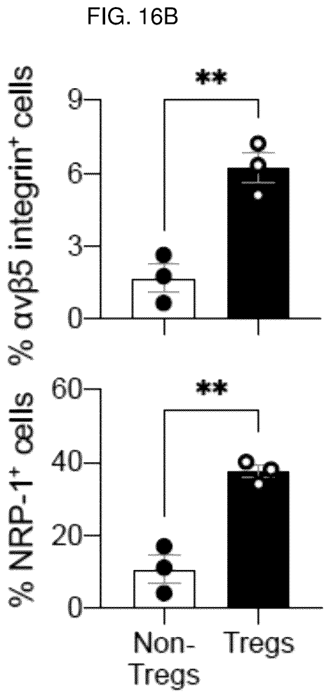

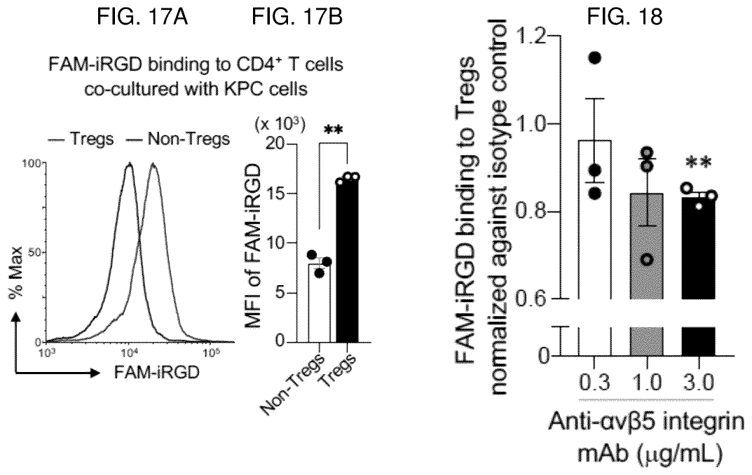

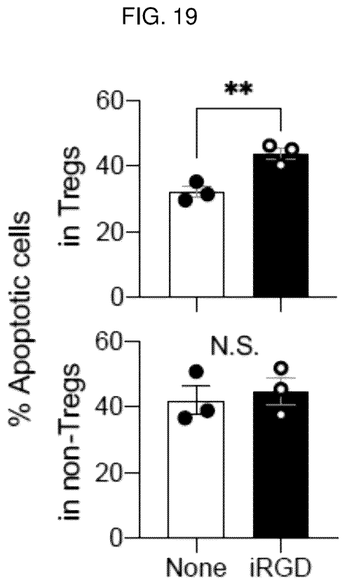

For the data presented in FIG. 16 through FIG. 19, CD4+ T cells isolated from the spleen of healthy C57B6129SF1/J hybrid mice were expanded for 3 days in vitro in the presence of KPC-derived PDAC cells. Flow cytometry was performed for subsequent analyses. FIG. 16A shows the expression of αvβ5 integrin and NRP-1 on CD4+CD25+ Tregs expanded with or without the PDAC cells (n=4 per group). FIG. 16B shows the expression of αvβ5 integrin and NRP-1 on CD4+CD25+ Tregs and non-Treg CD4+CD25neg T cells expanded with the PDAC cells (n=3 per group). FIG. 17A and FIG. 17B shows FAM-iRGD binding to the non-Tregs (blue line) and Tregs (red line) shown in FIG. 16B. The bar diagram summarizes the median fluorescence intensity (MFI) from 4 independent experiments. FIG. 18 shows the dose-dependent inhibition of FAMiRGD binding by an anti-αvβ5 integrin blocking Ab to Tregs that were expanded in the presence of PDAC cells. Values were normalized against isotype control (n=3 per group). Statistical analysis was performed between the isotype control and anti-αvβ5 integrin values. For the data in FIG. 19, Tregs and non-Tregs were expanded in the presence of PDAC cells with or without iRGD. Apoptosis was quantitated by measuring annexin V and 7-AAD double positive cells by flow cytometry (n=3).

The presence of PDAC cells greatly enhanced the induction of αvβ5 integrin+CD4+CD25+ Tregs (FIG. 16A, top panel). The Tregs consistently expressed NRP-1 regardless of the presence of PDAC cells (FIG. 16A, bottom panel). Non-Treg CD4+CD25neg T cells showed significantly less expression of αvβ5 integrin (and NRP-1) even in the presence of PDAC cells (FIG. 16B).

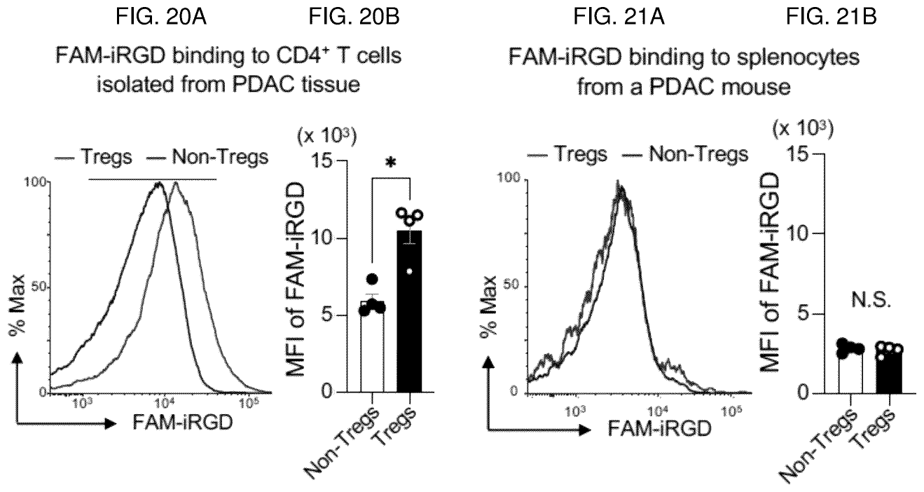

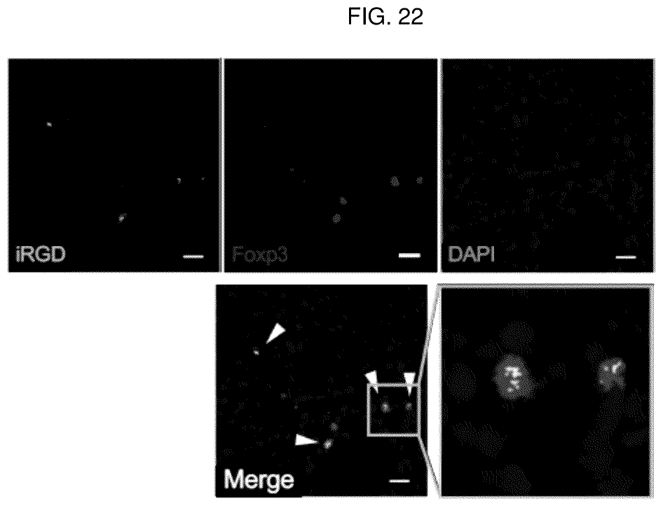

FIG. 20AB and FIG. 21A and FIG. 21B present data on in vitro binding of FAM-iRGD to non-Treg CD4+CD25neg T cells (blue line) and CD4+CD25+ Tregs (red line) isolated from the PDAC tissue (FIG. 20A and FIG. 20B) or the spleen (FIG. 21A and FIG. 21B) of KPC-derived orthotopic PDAC mice. The bar diagrams summarize the MFI from 4 independent experiments. FIG. 22 shows representative confocal images of Foxp3+ T cells (red) in the PDAC tissue of KPC-derived orthotopic PDAC mice that received an intravenous injection of FAM-iRGD (green). Blue=DAPI; arrows indicate Tregs positive for iRGD. The boxed area is magnified. Scale bars, 20 μm.

Statistical analyses used were Mann-Whitney U test (FIG. 16A, FIG. 17, FIG. 20, FIG. 21), Welch's t test (FIG. 16B and FIG. 19), and one sample Wilcoxon signed rank test (FIG. 18). p=0.0286 (FIG. 16A, top), p=0.3429 (FIG. 16A, bottom), p=0.0058 (FIG. 16B, top), p=0.0086 (FIG. 16B, bottom), p=0.0286 (FIG. 17), p=0.7272 (FIG. 18, 0.3), p=0.1766 (FIG. 18, 1.0), p=0.0052 (FIG. 18, 3.0), p=0.0087 (FIG. 19, top), p=0.6583 (FIG. 16B, bottom), p=0.0286 (FIG. 20), p=0.6857 (FIG. 21). Error bars show the mean standard error; *p<0.05; **p<0.01; N.S., not significant.

### Example 13: αvβ5 Integrin+ Tregs are Induced from Naïve CD4+ T Cells

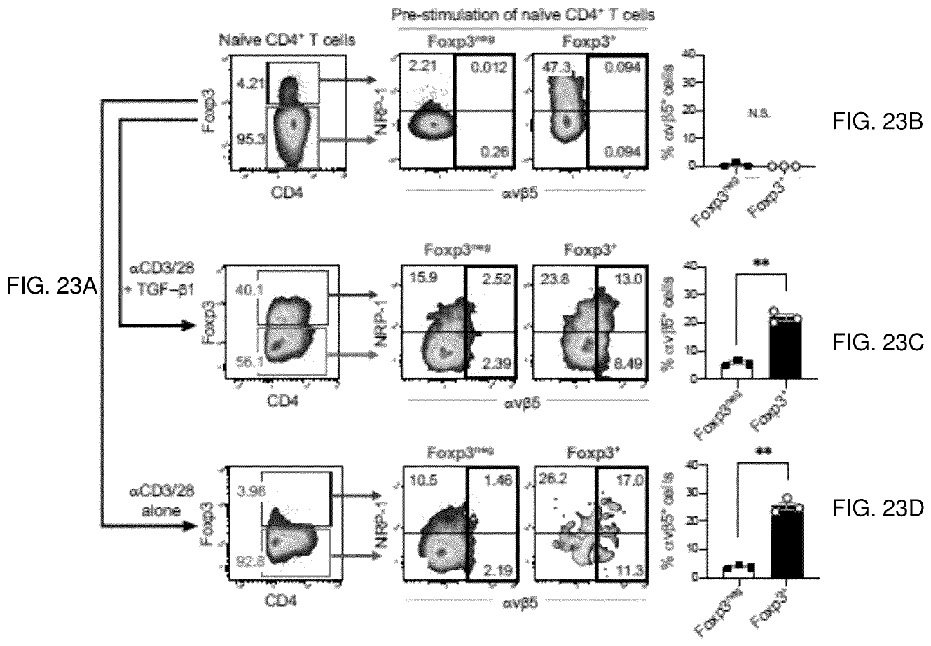

In general, tumor-resident Tregs are believed to originate from two major sources, induced Tregs (iTregs) that differentiate in the periphery from naïve CD4+ T cells and natural Tregs (nTregs) that develop in the thymus. To understand the origin of αvβ5 integrin+ Tregs, we first tested whether they can be induced as iTregs from naïve CD4+ T cells, which were magnetically enriched from mouse splenocytes (see FIG. 14A-23A, top row, and FIG. 23B).

Naïve CD4+ T cells were isolated from the spleen of healthy C57B6129SF1/J hybrid mice by magnetically removing CD4neg T cells and CD25+ T cells. The pool enriched for naïve CD4+ T cells was cultured in vitro with anti-CD3/CD28 Abs in the presence or absence of TGF-β1 for 3 days, and analyzed for αvβ5 integrin and NRP-1 expression by flow cytometry. See FIG. 23A, top row, and FIG. 23B.

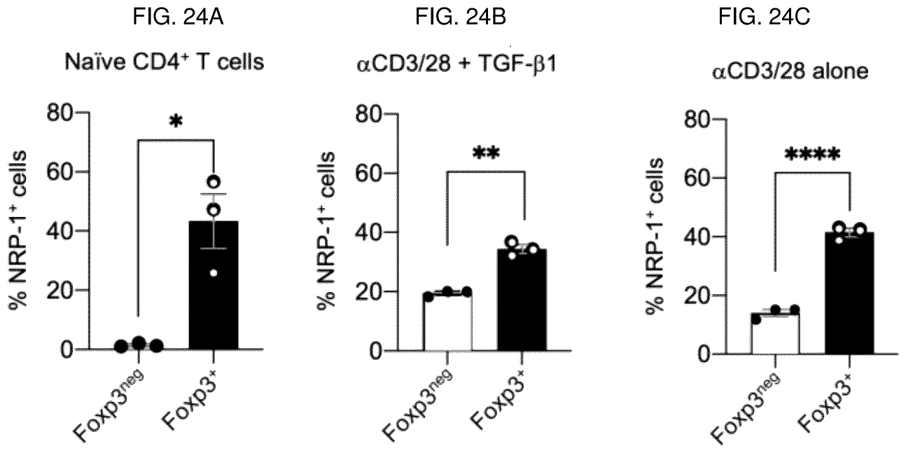

The pool contained a small number of CD4+Foxp3+ T cells, which were presumably nTregs that were not completely removed. In fact, they had high expression of NRP-1 compared to the naïve CD4+ T cells (see FIG. 24A). Both the naïve CD4+ T cells and CD4+Foxp3+ T cells lacked αvβ5 integrin expression. In line with previous reports, treating the pool with anti-CD3/CD28 bispecific Abs along with TGF-β1 led to the expansion of CD4+Foxp3+ iTregs (see FIG. 23A, middle row and FIG. 123C). Naïve CD4+ T cells (blue box) were enriched from mouse splenocytes. A minor population of CD4+Foxp3+ T cells was present (red box). See FIG. 23A, middle row, and FIG. 23C.

Over 20% of the resulting iTregs expressed the αvβ5 integrin, while only a minor population of CD4+Foxp3neg non-Tregs expressed the αvβ5 integrin. The iTregs had higher expression of NRP-1 than the non-Tregs (FIG. 24B). On the other hand, TCR stimulation alone did not lead to the development of iTregs from naïve CD4+ T cells evidenced by the fact that >90% of the cells remained negative for Foxp3 (FIG. 23A, bottom row, blue box, and FIG. 23D. Only 3-4% of the CD4+Foxp3neg T cells expressed the αvβ5 integrin. The results indicate that both TCR stimulation and TGF-β1 are required for the induction of iTregs from naïve CD4+ T cells, and that αvβ5 integrin+ Tregs arise as a subpopulation of the iTregs. Interestingly, some of the CD4+Foxp3+ T cells (FIG. 23A, bottom row, red box, and FIG. 23D that were initially negative for αvβ5 integrin were found to express the integrin after TCR stimulation alone, suggesting that nTregs may also be a potential source for αvβ5 integrin+ Tregs. Treating the pool in the top row with anti-CD3/CD28 Abs and TGF-β1 yielded approximately 40% of CD4+Foxp3+ T cells (red box) and 56% of CD4+Foxp3neg T cells (blue box), but treating the pool in the top row with anti-CD3/CD28 Abs alone did not change the proportion of the CD4+ T cells. Nearly 95% of the cells remained negative for Foxp3 (blue box). See FIG. 23A, bottom row, and FIG. 23D. A significant portion of the cells also expressed NRP-1 (see FIG. 24C).

Representative dot plots showing the proportion of CD4+Foxp3⁺ T cells (FIG. 23A, left panels) and the expression of αvβ5 integrin and NRP-1 on the indicated population are presented. The bar diagrams summarize the proportion of αvβ5+ cells in the indicated population (n=3 per study). For statistical analysis, Welch's t test; p=0.4115 (FIG. 23A, top), p=0.0013 (FIG. 23A, middle, and FIG. 23C), p=0.0032 (FIG. 23A, bottom, and FIG. 23D) were used. Error bars indicate the mean±standard error; **p<0.01; N.S., not significant.

For the data presented in FIG. 24A, NRP-1 expression on naive CD4+Foxp3neg T cells and CD4+Foxp3+ T cells prior to the treatment. FIG. 24B and FIG. 24C present data on NRP-1 expression on CD4+Foxp3neg T cells and CD4+Foxp3+ T cells after treatment with anti-CD3/CD28 Abs and TGF-β1 (FIG. 21B) or with anti-CD3/CD28 Abs alone (FIG. 24C) (n=3 per study). The statistical analysis used was Welch's t test; p=0.0435 (FIG. 24A), p=0.0027 (FIG. 24B), p<0.0001 (FIG. 24C). The error bars indicate the mean standard error; *p<0.05; **p<0.01; ****p<0.0001.

### Example 14: TGF-βR1 Inhibitor does not Affect the Expression of αvβ5 Integrin or NRP-1 on iTregs

To test whether TGF-β1 was an inducer of αvβ5 integrin+ iTregs based on the data that TGF-β1 on top of TCR stimulation was required for iTreg induction, and because TGF-β1 is a well-accepted inducer of integrin expression on various cells, a study was performed. Our recent data has showed that TGF-β1 secreted by CAFs (and PDAC cells) was an inducer of αvβ5 integrin expression on PDAC cells.

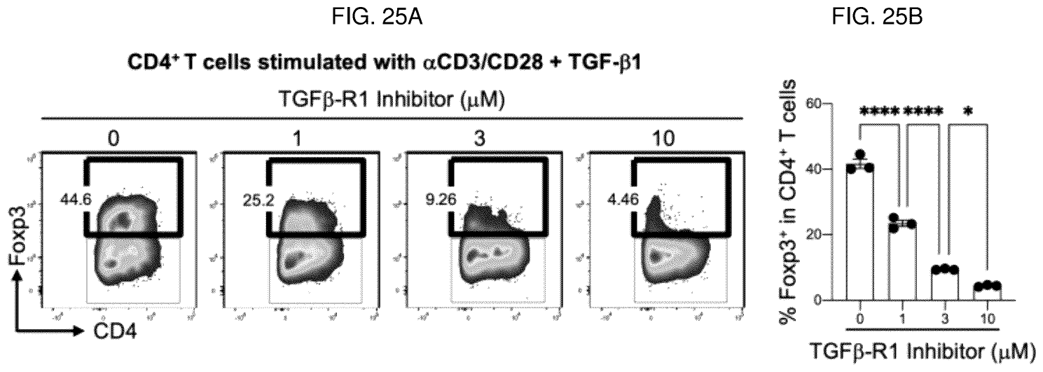

Naive CD4+ T cells isolated from healthy mice were expanded in the presence of anti-CD3/CD28 Abs, exogenous TGF-β1, and increasing doses of a TGF-βR1 inhibitor (LY2157299). FIG. 25A shows a flow cytometric analysis showing the proportion of CD4+Foxp3+ T cells (iTregs) after the expansion. The bar diagram of FIG. 25B summarizes the results from 3 independent experiments. The statistical analysis used was one-way ANOVA; p<0.0001 (0 vs 1), p<0.0001 (1 vs 3), p=0.0154 (3 vs 10).

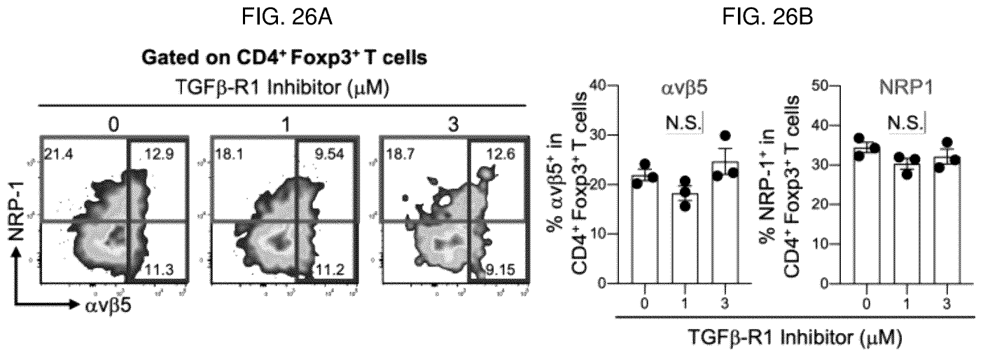

FIG. 26A shows the expression of αvβ5 integrin (red boxes) and NRP-1 (blue boxes) on the iTregs gated in FIG. 25A. The bar diagram of FIG. 26B summarizes the results from 3 independent experiments. The statistical analysis used was one-way ANOVA; p=0.3996 (αvβ5, 0 vs 1), p=0.5868 (αvβ5, 0 vs 3), p=0.2265 (NRP-1, 0 vs 1), p=0.5751 (NRP-1, 0 vs 3). The error bars indicate the mean±standard error; *p<0.05; ****p<0.0001; N.S., not significant.

As expected, LY2157299, an inhibitor of TGF-β receptor type 1 (TGF-βR1), suppressed the development of iTregs from naïve CD4+ T cells in a dose-dependent manner (FIG. 25A and FIG. 25B). However, we found that the inhibitor did not affect the proportion of the αvβ5-positive (or NRP-1-positive) iTregs (FIG. 26A and FIG. 26B). Thus, TGF-β1 is indeed a critical inducer of iTregs, but does not specifically induce the αvβ5-positive population among the iTregs.

### Example 15: αvβ5 Integrin Expression is Induced in Response to TCR Stimulation and Correlates with CD25 Expression

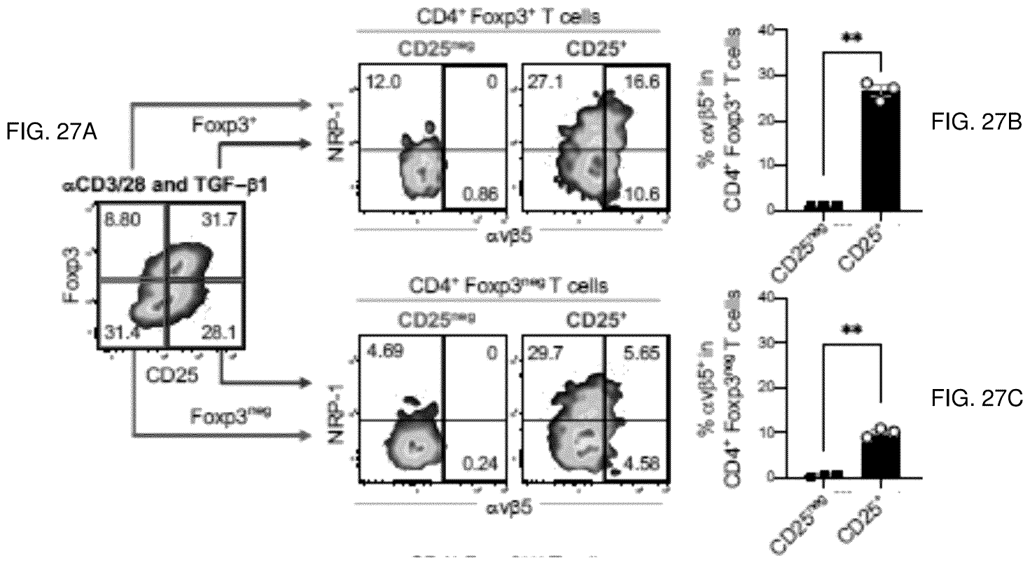

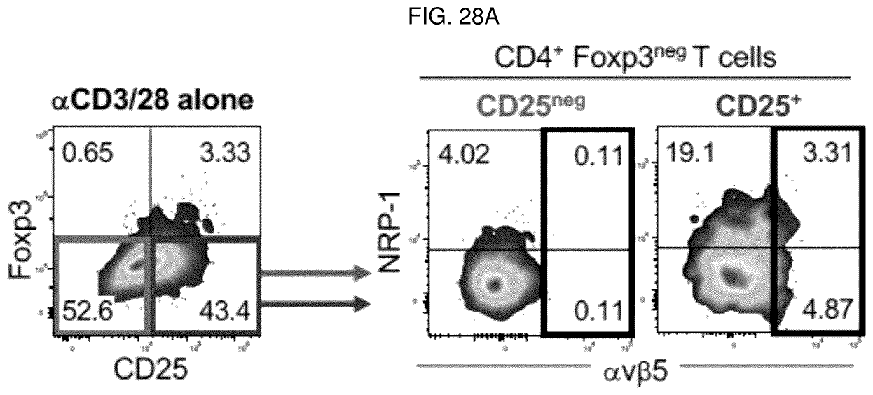

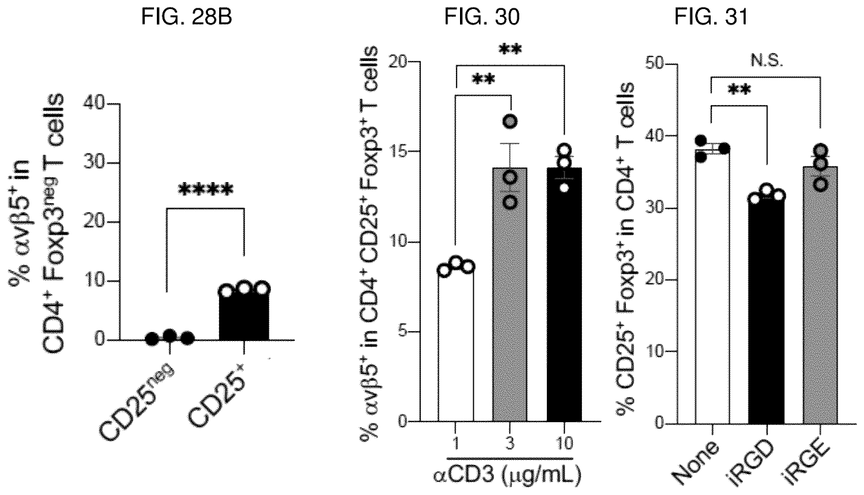

Further analysis revealed that αvβ5 integrin expression appeared to correlate with CD25, which is expressed on CD4+ T cells upon TCR stimulation. Naïve CD4+ T cells isolated from healthy mouse spleen were expanded in the presence of anti-CD3/CD28 Abs and TGF-β1 (see FIG. 27) or anti-CD3/CD28 Abs alone (see FIG. 28) for 3 days. The resulting populations were gated based on Foxp3 and CD25 expression (left panels). αvβ5 integrin and NRP-1 expression on Foxp3+ cells (FIG. 27A, top row) and Foxp3neg cells (FIG. 27A, bottom row and FIG. 28) were analyzed by flow cytometry. The red and blue boxes gate CD25+ and CD25neg cells, respectively. The bar diagrams summarize the proportion of αvβ5 integrin-positive cells in the indicated T cell population (n=3). Statistical analysis used was the Welch's t test; p=0.0021 (FIG. 27A, top, and FIG. 27B), p=0.0014 (FIG. 27A, bottom, and FIG. 27C), p<0.0001 (FIG. 28A).

Nearly 30% of the CD4+CD25+Foxp3+ iTregs that were induced from naïve CD4+ T cells by TCR stimulation and TGF-31 expressed the αvβ5 integrin, while almost none of the CD4+CD25^{neg} Foxp3+ T cells expressed the integrin (FIG. 27A, top row, and FIG. 27B). Although the proportion was significantly less than the iTregs, some of the CD4+CD25+Foxp3neg T cells derived from naïve CD4+ T cells also expressed the αvβ5 integrin (FIG. 27A, bottom row, and FIG. 27C). CD4+CD25neg Foxp3neg T cells remained negative for αvβ5 integrin expression.

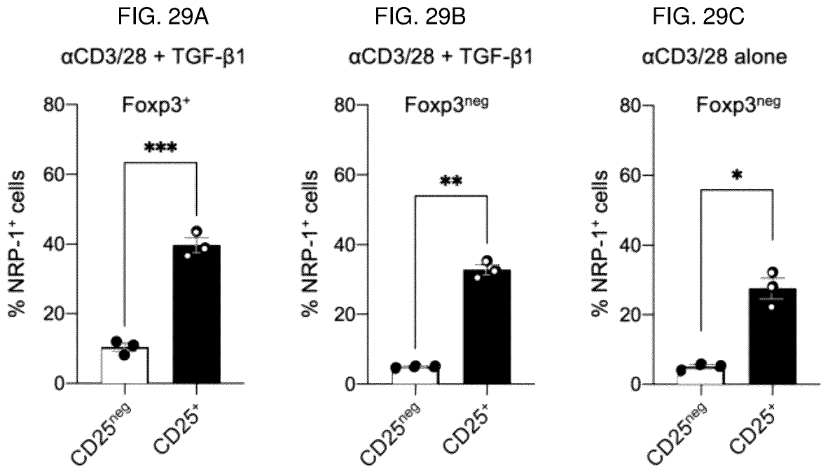

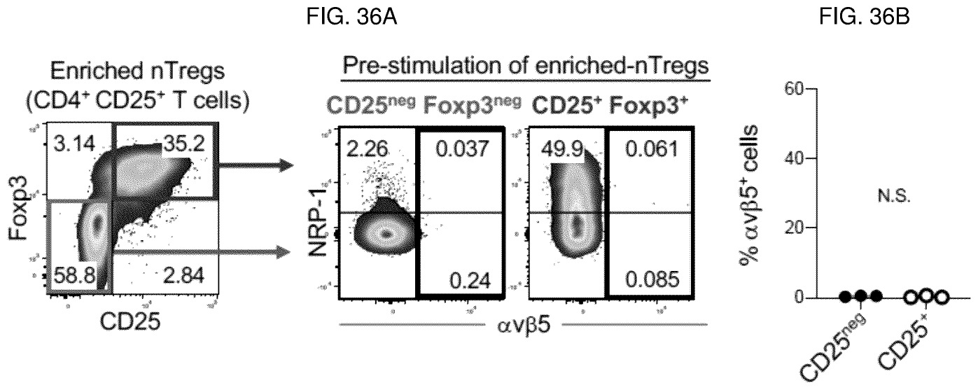

To examine the relevance of NRP-1 and CD25 expression profiles in naive CD4+ T cells that received TCR stimulation with or without TGF-β1. A pool of CD4+ T cells enriched for naive CD4+ T cells were treated with anti-CD3/CD28 Abs in the presence (see FIG. 29A and FIG. 29B) or absence (see FIG. 29C) of TGF-β1 and subjected to flow cytometry as described below for FIG. 36. The bar diagrams of FIG. 29 show the proportion of NRP-1+ cells among CD25-positive and negative populations in CD4+Foxp3+ T cells (FIG. 29A) and CD4+Foxp3neg T cells (FIG. 29B and FIG. 29C) after the treatment (n=3 per study).

Similar to the expression profile of the αvβ5 integrin, NRP-1 expression was significantly higher on CD25+ cells (see FIG. 29A and FIG. 29B). CD4+CD25+Foxp3neg T cells that developed from naïve CD4+ T cells with TCR stimulation alone also showed minor expression of the αvβ5 integrin (FIG. 28). The cells also expressed NRP-1 to some extent (see FIG. 29C).

The statistical analysis used was the two-tailed unpaired Welch's t test; p=0.001 (FIG. 29A), p=0.0021 (FIG. 29B), p=0.0135 (FIG. 29C). The error bars indicate the mean±standard error; *p<0.05; **p<0.01.

These results suggest that TCR stimulation is likely a key inducer of αvβ5 integrin (and NRP-1) expression on iTregs. FIG. 30 shows flow cytometric analysis showing the proportion of αvβ5 integrin+ cells among CD4+CD25+Foxp3+ iTregs induced by increasing concentrations of anti-CD3 Ab (n=3). The statistical analysis used here was one-way ANOVA; p=0.0088 (1 vs 3), p=0.0088 (1 vs 10). In fact, αvβ5 integrin+CD4+CD25+Foxp3+ iTregs were dose-dependently induced by an anti-CD3 Ab (FIG. 5C30), which provides TCR-mediated T cell activation signals.

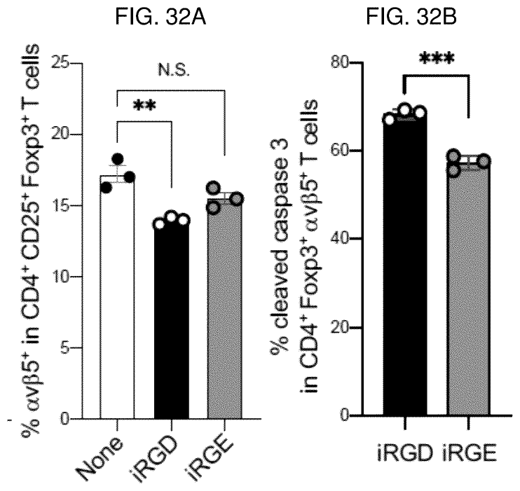

For the data presented in FIG. 31, FIG. 32A, and FIG. 32B, naïve CD4+ T cells were stimulated with anti-CD3/CD28 Abs and TGF-β1 in the absence or presence of iRGD or iRGE. Flow cytometry was performed to quantify the proportion of CD4+CD25+Foxp3+ iTregs (see FIG. 31) and αvβ5 integrin+ cells among the iTregs (See FIG. 32A). Apoptosis of αvβ5 integrin+ iTregs was quantitated by measuring cleaved caspase 3 using flow cytometry (see FIG. 32B; n=3).

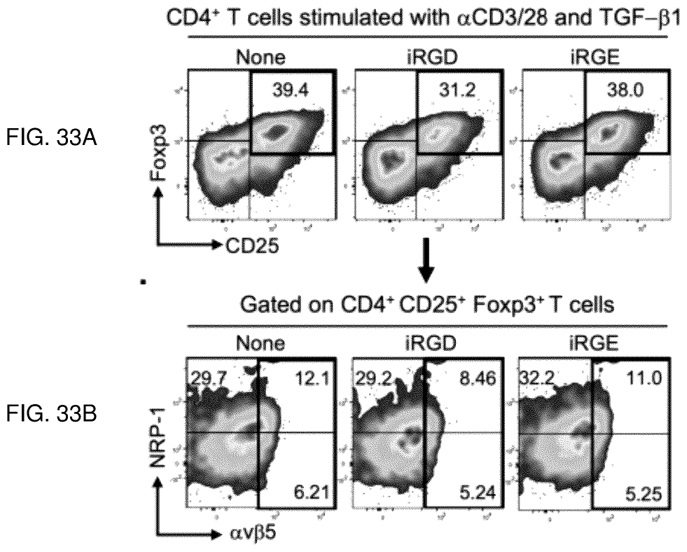

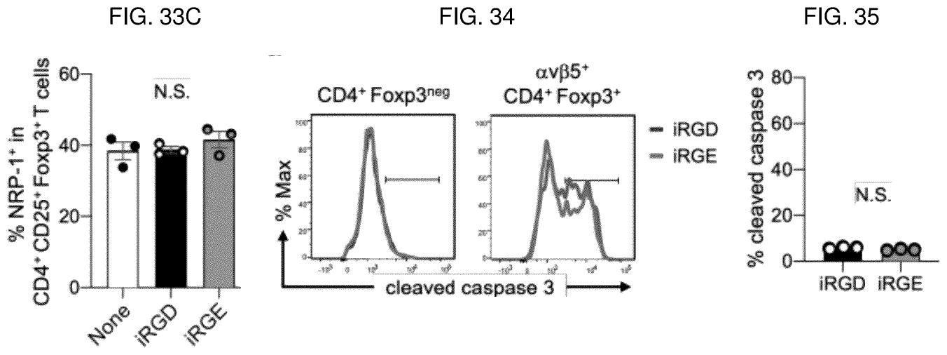

To investigate whether iRGD Reduces CD4+CD25+Foxp3+ iTregs in an RGD-Dependent Manner in Vitro, naïve CD4+ T cells were stimulated with anti-CD3/CD28 Abs and TGF-β1 in the absence or presence of the iRGD or iRGE peptide. The resulting cells were subjected to flow cytometry to analyze the expression of CD25 and Foxp3 in the CD4+ T cells (FIG. 33A) and the expression of the αvβ5 integrin and NRP-1 on CD4+CD25+Foxp3+ iTregs (FIG. 33B). The bar diagram in (FIG. 33C) summarizes the proportion of NRP-1+ cells among the iTregs. The statistical analysis used was the one-way ANOVA; p=0.8221. n=3 per group.

The proportion of apoptotic αvβ5 integrin+ iTregs (FIG. 34) and CD4+Foxp3neg T cells (FIG. 35) was quantitated by measuring cleaved caspase 3 using flow cytometry. The statistical analysis used was Welch's t test; p=0.0705 (n=3). The dot plots and histograms of FIG. 33C and FIG. 35 show representative data from the 3 independent experiments. The error bars indicate the mean±standard error; N.S., not significant.

In vitro iRGD treatment led to the reduction of CD4+CD25+Foxp3+ iTregs (FIG. 31 and FIG. 33A) and the αvβ5 integrin-positive population among the iTregs (FIG. 32A and FIG. 33B). The treatment did not alter the proportion of NRP-1-positive iTregs (FIG. 33C). The effect was dependent on its integrin-binding RGD motif because iRGE (CRGEKGPDC; SEQ ID NO:1), an iRGD variant that lacks the RGD motif, was ineffective. The reduction was in part due to increased apoptosis of αvβ5 integrin+ Tregs based on elevated levels of cleaved caspase 3 (FIG. 32B and FIG. 34). Neither iRGD nor iRGE increased the apoptosis of CD4+Foxp3neg T cells (FIG. 35).

Statistical analysis used here was one-way ANOVA (FIG. 31 and FIG. 5E-22-33-32A) or Welch's t test (FIG. 32B); p=0.0042 (FIG. 31, None vs iRGD), p=0.1774 (d, None vs. iRGE), p=0.0027 (FIG. 32A, None vs iRGD), p=0.0529 (FIG. 32A, None vs iRGE), p=0.001 (FIG. 32B). Error bars indicate the mean±standard error; *p<0.05; **p<0.01; ****p<0.0001; N.S., not significant.

### Example 16: NTregs Express αvβ5 Integrin in Response to TCR Stimulation

The findings in iTregs prompted us to explore whether TCR stimulation also induces αvβ5 integrin expression on nTregs. It seemed likely given our earlier finding that the minor CD4+Foxp3+ T cell population in the pool enriched for naïve CD4+ T cells turned positive for αvβ5 integrin upon TCR stimulation (refer to FIG. 23A, bottom row). Accordingly, we prepared a pool of CD4+ T cells enriched for nTregs using CD4 and CD25 as selection markers.

CD4+CD25+Foxp3+ T cells (nTregs) were enriched from the spleen of healthy C57B6129SF1/J hybrid mice by magnetically removing CD4neg T cells and CD25neg T cells. The two left panels of FIG. 36A show the expression of αvβ5 integrin and NRP-1 on the nTregs (red box) and naïve CD4+CD25neg Foxp3neg T cells (blue box) analyzed by flow cytometry. The bar diagram summarizes the proportion of αvβ5 integrin+ cells among the two populations (n=3). The statistical analysis used here was Welch's t test; p=0.4721.

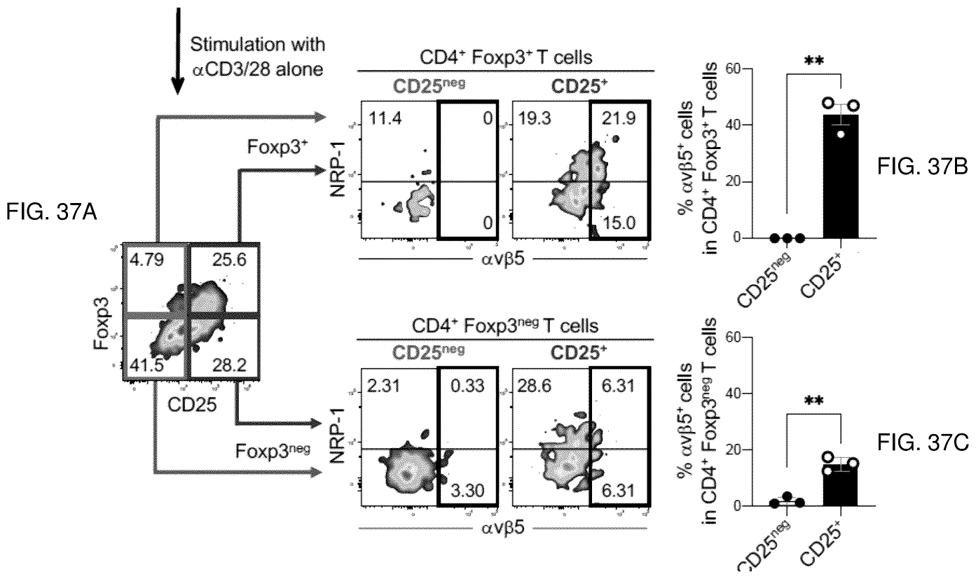

The pool in (FIG. 36A) was treated with anti-CD3/CD28 Abs alone for 3 days (FIG. 37A, left panel). αvβ5 integrin and NRP-1 expression on Foxp3+ cells (FIG. 37A, top row, and FIG. 37B) and Foxp3neg cells (FIG. 37A, bottom row, and FIG. 37C) was analyzed by flow cytometry. The red and blue boxes gate CD25+ and CD25neg cells, respectively. The bar diagrams summarize the proportion of αvβ5 integrin+ cells among the indicated T cell populations (n=3). The statistical analysis used was Welch's t test; p=0.0064 (FIG. 37A, top, and 37B), p=0.0035 (FIG. 37A, bottom, and FIG. 37C). Error bars indicate the mean standard error; **p<0.01; N.S., not significant.

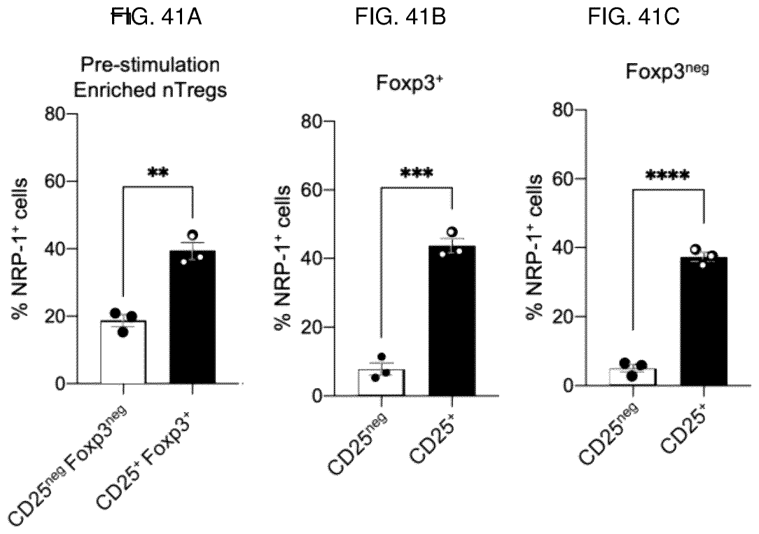

Approximately 35% was CD4+CD25+Foxp3+ nTregs in the resulting pool (FIG. 36A), which was nearly 10 times more than that in the pool enriched for naïve CD4⁺ T cells (refer to FIG. 23). Both the nTregs and the remaining naïve CD4+CD25neg Foxp3neg T cells were negative for the αvβ5 integrin. The nTregs expressed NRP-1 as expected (see FIG. 41A). Treating the pool with anti-CD3/CD28 Abs led to the induction of αvβ5 integrin+CD4+CD25+Foxp3+ T cells (FIG. 37A, top row). These cells were most likely nTregs, and not iTregs that were induced from naïve CD4+ T cells, because anti-CD3/CD28 Abs alone did not induce iTregs under the same condition (refer to FIG. 23A, bottom row).

The expression of αvβ5 integrin was noted exclusively on nTregs, and not on CD4+CD25neg Foxp3+ T cells, which act as peripheral reservoirs of differentiated nTregs that become CD25-positive upon expansion or activation. Consistent with our earlier data, CD4+CD25+Foxp3neg T cells, which likely expanded from naïve CD4⁺ T cells upon TCR stimulation, expressed low levels of αvβ5 integrin (FIG. 37A, bottom row). NRP-1 expression was also mainly restricted to CD25+ cells (see also FIG. 41B and FIG. 41C).

These results suggest that αvβ5 integrin expression can be induced on nTregs through TCR stimulation. While the mode of αvβ5 integrin and CD25 expression correlates to some extent, it does not completely match because the CD25+ nTregs prior to TCR stimulation did not express the αvβ5 integrin. Thus, taken together with the iTreg data, the data suggests that αvβ5 integrin can serve as an activation marker for Tregs that received TCR stimulation in the periphery.

### Example 17: The αvβ5 Integrin Marks a Highly Immunosuppressive Subpopulation of CCR8+ Tregs

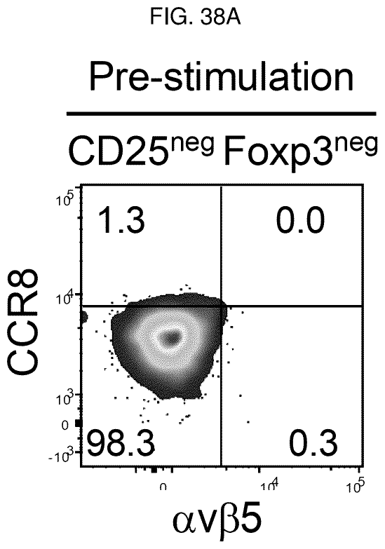

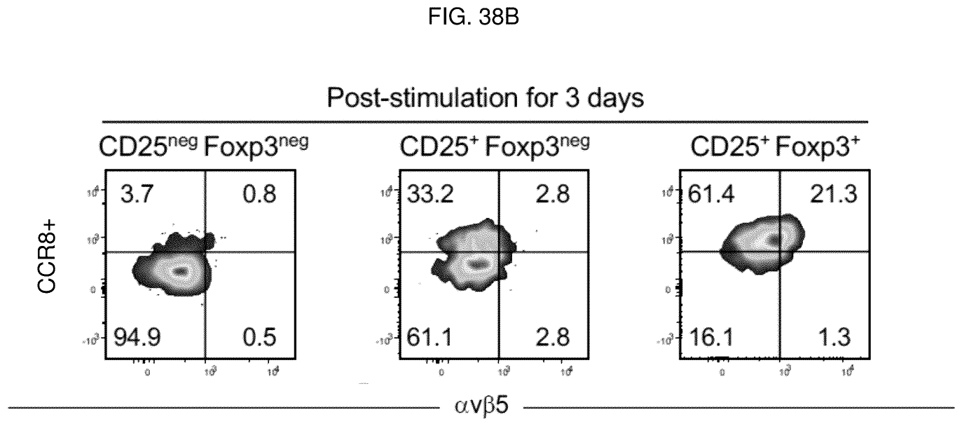

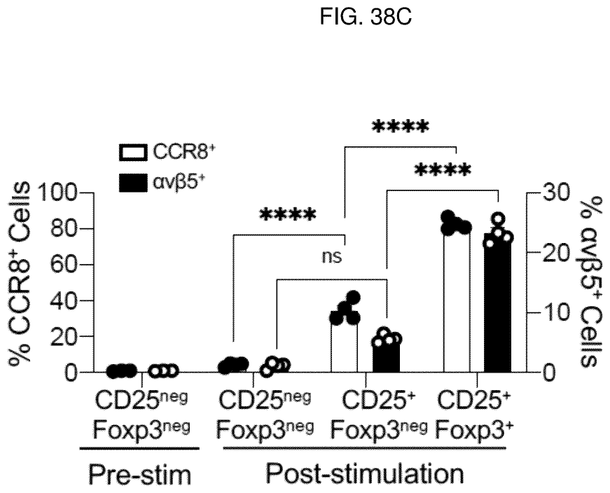

CCR8 is one of the few tumor-specific activation markers of Tregs. Targeting CCR8 leads to tumor-specific depletion of Tregs and enhanced tumor-specific immunity, which resembles the outcome iRGD therapy achieves in PDAC mice. We therefore hypothesized that the αvβ5 integrin has a similar expression profile as CCR8. Flow cytometry showed that naïve CD4+CD25neg Foxp3neg T cells did not express CCR8 (or αvβ5 integrin) (see FIG. 38A).

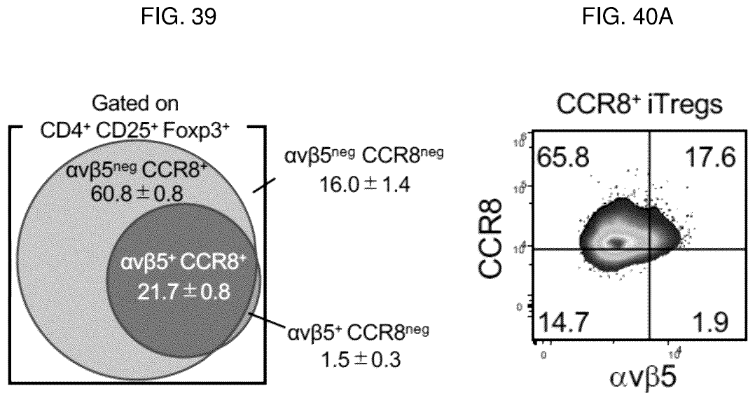

Naïve CD4+ T cells were magnetically isolated from the spleen of healthy C57B6129SF1/J hybrid mice. The pool enriched for naïve CD4+ T cells was cultured in vitro in the presence of anti-CD3/CD28 Abs and TGF-β1 for 3 days. FIG. 38A and FIG. 38B show the expression of CCR8 and αvβ5 integrin on CD4+CD25neg Foxp3neg T cells, CD4+CD25+Foxp3neg T cells before and after stimulation. CD4+CD25+Foxp3+ iTregs were analyzed by flow cytometry. Representative dot plots from 3 separate studies are shown. FIG. 39 is a Venn diagram summarizing the proportion of Tregs that express CCR8 and/or αvβ5 integrin based on the results from FIG. 38.

Treating the cells with anti-CD3/CD28 Abs and TGF-β1 led to the induction of CCR8⁺ CD4⁺ CD25⁺ Foxp3⁺ iTregs (see FIG. 38A and FIG. 38B). αvβ5 integrin was expressed on approximately 25% of the CCR8+ iTregs and nearly all the αvβ5 integrin+ iTregs expressed CCR8, indicating that αvβ5 integrin expression was restricted to a subpopulation of CCR8+ iTregs (see FIG. 39). Approximately 30% of CD4+CD25+Foxp3neg T cells expressed CCR8, but the cells were largely negative for αvβ5 integrin. CD4+CD25neg Foxp3neg T cells remained negative for both CCR8 and αvβ5 integrin. FIG. 38C summarizes the proportion of CCR8+ cells (white bars) and αvβ5 integrin+ cells (black bars) among each T cell population in FIG. 38A and FIG. 38B.

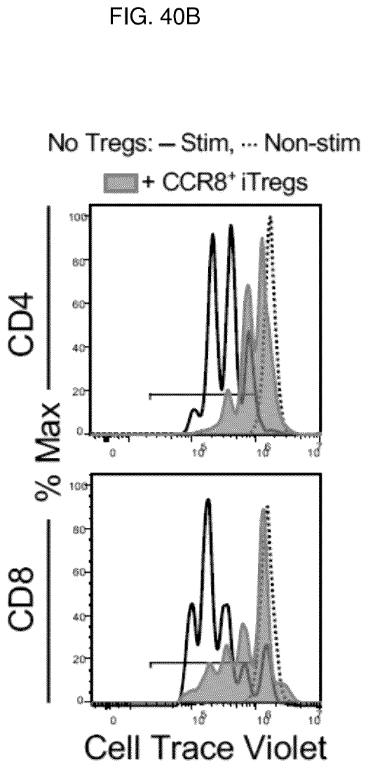

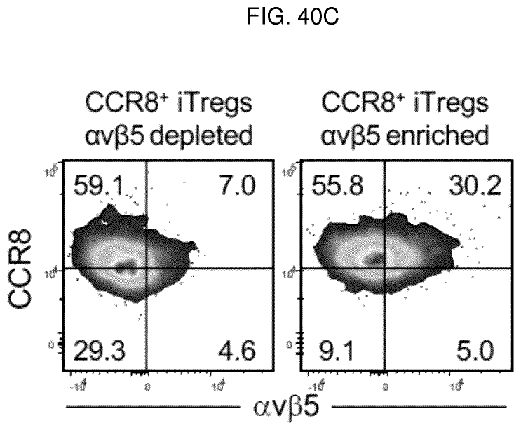

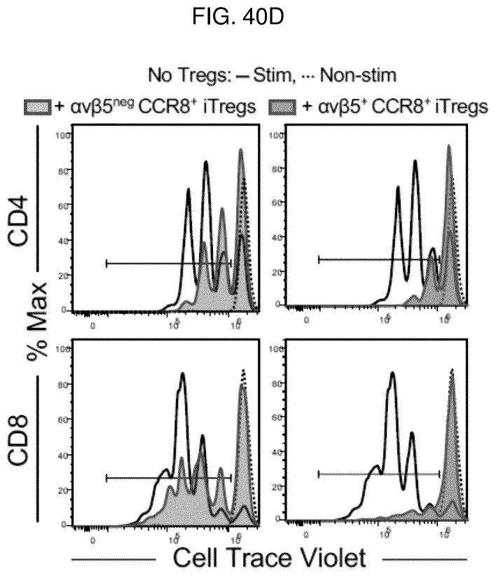

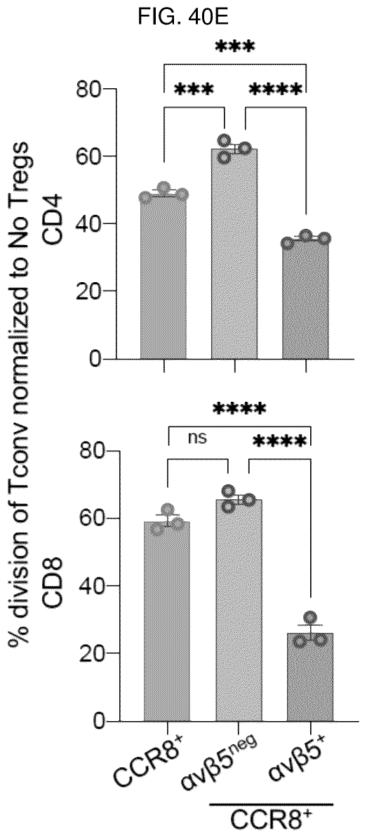

These findings led us to hypothesize that the αvβ5 integrin defines a functionally distinct population of CCR8+ Tregs that have received TCR stimulation. To test this hypothesis, we induced iTregs from mouse naïve CD4⁺ T cells, and magnetically enriched for CCR8⁺ iTregs (FIG. 40A). Immunosuppression assays were performed by co-culturing iTregs and Tconv (CD4⁺ and CD8⁺) at a 1:4 ratio in the presence of anti-CD3/CD28 Abs (TCR stimulation) for 3 days. We used iTregs that were enriched for CCR8+ iTregs (FIG. 40A and FIG. 40B) or CCR8+ iTregs that were either depleted or enriched for αvβ5 integrin+ cells (FIG. 40C and FIG. 40D). Immunosuppression assays were performed with or without a pool of iTregs enriched for CCR8+ iTregs (see FIG. 40A and FIG. 40B) or αvβ5 integrin+ CCR8+ iTregs and αvβ5 integrin neg CCR8+ iTregs (see FIG. 40C). The expression of Foxp3, CCR8, and αvβ5 integrin is shown in the representative dot plots. The Tregs were mixed with Tconv (CD4+ and CD8+) at a ratio of 1:4. Proliferation of Tconv was analyzed by flow cytometry using CellTrace™ Violet as shown in the representative histograms. The bar diagrams summarize the results (n=3). The statistical analysis used for the data in FIG. 38 through FIG. 40 was one-way ANOVA. Error bars indicate the mean±standard error; ***p<0.001; ****p<0.0001.

The pool effectively suppressed the proliferation of conventional CD4⁺ and CD8⁺ T cells (Tconv). To study the contribution of αvβ5 integrin+ Tregs in this effect, we prepared pools of CCR8⁺ iTregs that were either magnetically enriched or depleted for αvβ5 integrin+ iTregs (FIG. 40C and FIG. 40D). Enriching the αvβ5 integrin⁺ population led to a strikingly potent ability to suppress Tconv proliferation. The effect appeared to be more pronounced against CD8+ T cells than CD4+ T cells. In contrast, depleting αvβ5 integrin+ cells reduced the suppression properties. These results strongly suggest that αvβ5 integrin+ Tregs are highly immunosuppressive, and that they are the functionally dominant fraction of CCR8⁺ Tregs.

### Example 18: NRP-1 Expression on nTregs Before and After TCR Stimulation

A pool of CD4+ T cells enriched for CD4+CD25+Foxp3+ nTregs were treated with anti-CD3/CD28 Abs and subjected to flow cytometry as described for the data of FIG. 40 through FIG. 40. NRP-1 expression on the nTregs and CD4+CD25neg Foxp3neg T cells prior to the treatment is shown in FIG. 41A.

NRP-1 expression on CD25-positive and negative populations in CD4+Foxp3+ T cells (see FIG. 41A) and CD4+Foxp3neg T cells (see FIG. 41B) were analyzed after the treatment (n=3 per study). The statistical analysis used was Welch's t test; p=0.0028 (FIG. 41A), p=0.0002 (FIG. 41B), p<0.0001 (FIG. 41C). The error bars indicate the mean standard error; **p<0.01; ***p<0.001; ****p<0.0001.

### REFERENCES

All references listed below and throughout the specification are hereby incorporated by reference in their entirety for background and supporting information.
1. WO 2022/120344
2. Rucki A. A., Zheng L. Pancreatic cancer stroma: understanding biology leads to new therapeutic strategies. World J. Gastroenterol. 20, 2237-2246 (2014).
3. Sugiyama D. et al. Anti-CCR4 mAb selectively depletes effector-type FoxP3+CD4+ regulatory T cells, evoking antitumor immune responses in humans. Proc. Natl Acad. Sci. USA. 110, 17945-17950 (2013).
4. Muller M. et al. The Immune Landscape of Human Pancreatic Ductal Carcinoma: Key Players, Clinical Implications, and Challenges. Cancers (Basel) 14, (2022).
5. Nejati R. et al. Prognostic Significance of Tumor-Infiltrating Lymphocytes in Patients With Pancreatic Ductal Adenocarcinoma Treated With Neoadjuvant Chemotherapy. Pancreas 46, 1180-1187 (2017).
6. Tumeh P. C. et al. PD-1 blockade induces responses by inhibiting adaptive immune resistance. Nature 515, 568-571 (2014).
7. Aida K. et al. Suppression of Tregs by anti-glucocorticoid induced TNF receptor antibody enhances the antitumor immunity of interferon-alpha gene therapy for pancreatic cancer. Cancer Sci. 105, 159-167 (2014).
8. Arce Vargas F. et al. Fc-Optimized Anti-CD25 Depletes Tumor-Infiltrating Regulatory T Cells and Synergizes with PD-1 Blockade to Eradicate Established Tumors. Immunity 46, 577-586 (2017).
9. Jacobs C. et al. An ISCOM vaccine combined with a TLR9 agonist breaks immune evasion mediated by regulatory T cells in an orthotopic model of pancreatic carcinoma. Int. J. Cancer 128, 897-907 (2011).
10. Leach D. R., Krummel M. F., Allison J. P. Enhancement of antitumor immunity by CTLA-4 blockade. Science 271, 1734-1736 (1996).
11. Chen B. J., Zhao J. W., Zhang D. H., Zheng A. H., Wu G. Q. Immunotherapy of Cancer by Targeting Regulatory T cells. Int. Immunopharmacol. 104, 108469 (2022).
12. Tanaka A., Sakaguchi S. Regulatory T cells in cancer immunotherapy. Cell Res. 27, 109-118 (2017).
13. Di Giacomo A. M., Biagioli M., Maio M. The emerging toxicity profiles of anti-CTLA-4 antibodies across clinical indications. Semin. Oncol. 37, 499-507 (2010).
14. Wang H. et al. CD36-mediated metabolic adaptation supports regulatory T cell survival and function in tumors. Nat. Immunol. 21, 298-308 (2020).
15. Zhang Y. et al. Regulatory T-cell Depletion Alters the Tumor Microenvironment and Accelerates Pancreatic Carcinogenesis. Cancer Discov. 10, 422-439 (2020).
16. Kidani Y. et al. CCR8-targeted specific depletion of clonally expanded Treg cells in tumor tissues evokes potent tumor immunity with long-lasting memory. Proc. Natl Acad. Sci. USA. 119, (2022).
17. Weaver J. D. et al. Differential expression of CCR8 in tumors versus normal tissue allows specific depletion of tumor-infiltrating T regulatory cells by GS-1811, a novel Fc-optimized anti-CCR8 antibody. Oncoimmunology 11, 2141007 (2022).
18. Barsheshet Y. et al. CCR8(+)FOXp3(+) T(reg) cells as master drivers of immune regulation. Proc. Natl Acad. Sci. USA. 114, 6086-6091 (2017).
19. Plitas G. et al. Regulatory T Cells Exhibit Distinct Features in Human Breast Cancer. Immunity 45, 1122-1134 (2016).
20. Van Damme H. et al. Therapeutic depletion of CCR8(+) tumor-infiltrating regulatory T cells elicits antitumor immunity and synergizes with anti-PD-1therapy. J Immunother Cancer 9, (2021).
21. Sugahara K. N. et al. Tissue-penetrating delivery of compounds and nanoparticles into tumors. Cancer Cell 16, 510-520 (2009).
22. Teesalu T., Sugahara K. N., Kotamraju V. R., Ruoslahti E. C-end rule peptides mediate neuropilin-1-dependent cell, vascular, and tissue penetration. Proc. Natl Acad. Sci. USA. 106, 16157-16162 (2009).
23. Pang H. B. et al. An endocytosis pathway initiated through neuropilin-1 and regulated by nutrient availability. Nat Commun 5, 4904 (2014).
24. Sugahara K. N. et al. Coadministration of a tumor-penetrating peptide enhances the efficacy of cancer drugs. Science 328, 1031-1035 (2010).
25. Hurtado de Mendoza T. et al. Tumor-penetrating therapy for beta5 integrin-rich pancreas cancer. Nat Commun 12, 1541 (2021).
26. Uhlen M. et al. Proteomics. Tissue-based map of the human proteome. Science 347, 1260419 (2015).
27. Dean A., Gill S., McGregor M., Broadbridge V., Jarvelainen H. A., Price T. DualaV-integrin and neuropilin-1 targeting peptide CEND-1 plus nab-paclitaxel and gemcitabine for the treatment of metastatic pancreatic ductal adenocarcinoma: a first-in-human, open-label, multicentre, phase 1 study. Lancet Gastroenterol Hepatol 7, 943-951 (2022).
28. Bruder D. et al. Neuropilin-1: a surface marker of regulatory T cells. Eur. J. Immunol. 34, 623-630 (2004).
29. Hori S., Nomura T., Sakaguchi S. Control of regulatory T cell development by the transcription factor Foxp3. Science 299, 1057-1061 (2003).
30. Sakaguchi S., Wing K., Miyara M. Regulatory T cells—a brief history and perspective. Eur. J Immunol. 37 Suppl 1, S116-123 (2007).
31. Sugahara K. N. et al. Tumor-penetrating iRGD peptide inhibits metastasis. Mol. Cancer Ther. 14, 120-128 (2015).
32. Hansen W. et al. Neuropilin 1 deficiency on CD4+Foxp3+ regulatory T cells impairs mouse melanoma growth. J. Exp. Med. 209, 2001-2016 (2012).
33. Curotto de Lafaille M. A., Lafaille J. J. Natural and adaptive foxp3+ regulatory T cells: more of the same or a division of labor? Immunity 30, 626-635 (2009).
34. Sakaguchi S. Naturally arising CD4+ regulatory t cells for immunologic selftolerance and negative control of immune responses. Annu. Rev. Immunol. 22, 531-562 (2004).
35. Chen W. et al. Conversion of peripheral CD4+CD25− naive T cells to CD4+CD25+ regulatory T cells by TGF-beta induction of transcription factor Foxp3. J. Exp. Med. 198, 1875-1886 (2003).
36. Heino J., Ignotz R. A., Hemler M. E., Crouse C., Massague J. Regulation of Cell Adhesion Receptors by Transforming Growth Factor-P. J. Biol. Chem. 264, 380-388 (1989).
37. Ignotz R. A., Heino J., Massagu\tilde{e} J. Regulation of Cell Adhesion Receptors by Transforming Growth Factor-β. J. Biol. Chem. 264, 389-392 (1989).
38. Sheppard D., Cohen D. S., Wang A., Busk M. Transforming growth factor beta differentially regulates expression of integrin subunits in guinea pig airway epithelial cells. J. Biol. Chem. 267, 17409-17414 (1992).
39. Zambruno G. et al. Transforming growth factor-beta 1 modulates beta 1 and beta 5 integrin receptors and induces the de novo expression of the alpha v beta 6 heterodimer in normal human keratinocytes: implications for wound healing. J. Cell Biol. 129, 853-865 (1995).
40. Smith-Garvin J. E., Koretzky G. A., Jordan M. S. T cell activation. Annu. Rev. Immunol. 27, 591-619 (2009).
41. Shevach E. M. CD4+CD25+ suppressor T cells: more questions than answers. Nat. Rev. Immunol. 2, 389-400 (2002).
42. Zelenay S., Lopes-Carvalho T., Caramalho I., Moraes-Fontes M. F., Rebelo M., Demengeot J. Foxp3+CD25− CD4 T cells constitute a reservoir of committed regulatory cells that regain CD25 expression upon homeostatic expansion. Proc. Natl Acad. Sci. USA. 102, 4091-4096 (2005).
43. Nishikawa H., Sakaguchi S. Regulatory T cells in cancer immunotherapy. Curr. Opin. Immunol. 27, 1-7 (2014).
44. Ha T. Y. The role of regulatory T cells in cancer. Immune Netw. 9, 209-235 (2009).
45. Freeman Z. T. et al. A conserved intratumoral regulatory T cell signature identifies 4-1BB as a pan-cancer target. J Clin. Invest. 130, 1405-1416 (2020).
46. Okla K., Farber D. L., Zou W. Tissue-resident memory T cells in tumor immunity and immunotherapy. J. Exp. Med. 218, (2021).
47. Ahn E. et al. Role of PD-1 during effector CD8 T cell differentiation. Proc. Natl Acad. Sci. USA. 115, 4749-4754 (2018).
48. Hashimoto M. et al. CD8 T Cell Exhaustion in Chronic Infection and Cancer: Opportunities for Interventions. Annu. Rev. Med. 69, 301-318 (2018).
49. Ding N. et al. iRGD synergizes with PD-1 knockout immunotherapy by enhancing lymphocyte infiltration in gastric cancer. Nat Commun 10, 1336 (2019).
50. Hall B. M. et al. Do Natural T Regulatory Cells become Activated to Antigen Specific T Regulatory Cells in Transplantation and in Autoimmunity? Front. Immunol. 4, 208 (2013).
51. Thornton A. M., Shevach E. M. CD4+CD25+ immunoregulatory T cells suppress polyclonal T cell activation in vitro by inhibiting interleukin 2 production. J. Exp. Med 188, 287-296 (1998).
52. Ferreira L. M. R., Muller Y. D., Bluestone J. A., Tang Q. Next-generation regulatory T cell therapy. Nat Rev Drug Discov 18, 749-769 (2019).
53. Pytela R., Pierschbacher M. D., Ruoslahti E. A 125/115-kDa cell surface receptor specific for vitronectin interacts with the arginine-glycine-aspartic acid adhesion sequence derived from fibronectin. Proc. Natl Acad. Sci. USA. 82, 5766-5770 (1985).
54. Alavi A., Hood J. D., Frausto R., Stupack D. G., Cheresh D. A. Role of Raf in vascular protection from distinct apoptotic stimuli. Science 301, 94-96 (2003).
55. Hood J. D., Frausto R., Kiosses W. B., Schwartz M. A., Cheresh D. A. Differential alphav integrin-mediated Ras-ERK signaling during two pathways of angiogenesis. J. Cell Biol. 162, 933-943 (2003).
56. Li K. et al. Myeloid-derived suppressor cells as immunosuppressive regulators and therapeutic targets in cancer. Signal Transduct Target Ther 6, 362 (2021).
57. Poh A. R., Ernst M. Tumor-Associated Macrophages in Pancreatic Ductal Adenocarcinoma: Therapeutic Opportunities and Clinical Challenges. Cancers (Basel) 13, (2021).
58. Siret C. et al. Deciphering the Crosstalk Between Myeloid-Derived Suppressor Cells and Regulatory T Cells in Pancreatic Ductal Adenocarcinoma. Front. Immunol. 10, 3070 (2019).
59. Luheshi N. M. et al. Transformation of the tumour microenvironment by a CD40 agonist antibody correlates with improved responses to PD-L1 blockade in a mouse orthotopic pancreatic tumour model. Oncotarget 7, 18508-18520 (2016).

1. A method of obtaining a highly immunosuppressive Treg population comprising:
(a) obtaining a tumor sample from a subject; and
(b) isolating from the tumor sample CCR8+ Treg cells that express αvβ5 integrin.

2. The method of claim 1 wherein the tumor sample is a pancreatic tumor sample.

3. A method of obtaining a highly immunosuppressive Treg population comprising:
(a) obtaining a sample of CD4+ cells from a subject;
(b) contacting the CD4+ cells to a reagent selected from the group consisting of one or more of anti-CD3 antibody, anti-CD28 antibody, anti-CD3/CD28 bispecific antibody and TGFβ1 under conditions to bind a population of CD4+Foxp3+ Tregs; and
(c) isolating the CD4+Foxp3+ Tregs from the population of cells,
wherein the CD4+Foxp3+ Tregs express αvβ5 integrin.

4. The method of claim 3, wherein the isolated CD4+Foxp3+ Tregs are CCR8+ cells that express αvβ5 integrin.

5. An isolated population of αvβ5 integrin expressing CCR8+ Treg cells.

6. An isolated population of αvβ5 integrin expressing CCR8+ Treg cells obtained by the method of claim 1.

7. An isolated population of αvβ5 integrin expressing CD4+Foxp3+ Tregs obtained by the method of claim 3.

8. A method for reducing immune responses in a subject in need, the method comprising administering to the subject an effective composition comprising αvβ5 integrin expressing CCR8+ Treg cells of claim 5.

9. A method for reducing immune responses in a subject in need, the method comprising administering to the subject an effective composition comprising αvβ5 integrin expressing CCR8+ Treg cells of claim 6.

10. The method of claim 8, wherein the subject in need is a subject who has symptoms of an autoimmune disorder or has received a tissue transplant.

11. The method of claim 10, wherein the subject in need is experiencing graft-versus-host disease.

12. The method of claim 9, further comprising exposing a graft material to αvβ5 integrin expressing CCR8+ Treg cells prior to transplantation of the graft material.

13. A method of treating cancer in a subject in need, comprising: administering to the subject in need an αvβ5 integrin inhibitor in an amount effective to reduce αvβ5 integrin expression in the subject in need.

14. The method of claim 13, wherein the αvβ5 integrin inhibitor comprises an αvβ5 integrin specific antibody, an αvβ5 affinity probe, an αvβ5 integrin specific aptamer, a small molecule αvβ5 integrin inhibitor, or an αvβ5 integrin inhibitory oligonucleotide.

15. The method of claim 13, further comprising co-administering one or more immune checkpoint inhibitors to the subject in need.

16. The method of claim 15, wherein the one or more immune checkpoint inhibitors are selected from the group consisting of a PD-1 inhibitor, a PD-L1 inhibitor, a PD-L2 inhibitor, or any combination thereof.
